SEQUENCE LISTING (48 sequences)

SEQ ID NO: 1 - RNA, 63 nt, Schistosoma mansoni
ccuaaccuaugguccuggugagucccaaauaggacgaaacgccggcgucgaccaucauguagg

SEQ ID NO: 2 - RNA, 99 nt, Artificial sequence
  Synthetic sequence
gcggccgccugagaugcagguacauccagcugaugagucccaaauaggacgaaacgcgcuucggugcguccuggauuccacugcuauccacgcggccgc

SEQ ID NO: 3 - RNA, 97 nt, Artificial sequence
  Artificial sequence   Synthetic sequence
ccgcggcugagaugcagguacaucccacugaugagucccaaauaggacgaaacgcgcuucggugcgucugggauuccacugcuauccacgcggccgc

SEQ ID NO: 4 - RNA, 99 nt, Artificial sequence
  Synthetic sequence
gcggccgccugaggugcagguacauccagcugacgagucccaaauaggacgaaacgcgcuucggugcguccuggauuccacugcuauccacgcggccgc

SEQ ID NO: 5 - RNA, 83 nt, Artificial sequence
  Synthetic sequence
cugaggugcagguacauccagcugacgagucccaaauaggacgaaacgcgcuucggugcguccuggauuccacugcuauccac

SEQ ID NO: 6 - RNA, 64 nt, Satellite tobacco ringspot virus
gggagcccugucaccggaugugcuuuccggucugaugaguccgugaggacgaaacagggcuccc

SEQ ID NO: 7 - RNA, 54 nt, Artificial sequence
  Synthetic sequence
gcgcgucaccggaugugcuuuccggucugaugaguccgugaggaugaaacgcgc

SEQ ID NO: 8 - RNA, 89 nt, Artificial sequence
  Synthetic sequence
accggugcguccuggauuccacugcuauccauucgugaggugcagguacauccagcugacgagucccaaauaggacgaaacgcgccggu

SEQ ID NO: 9 - RNA, 81 nt, Artificial sequence
  Synthetic sequence
gcgcguccuggauuccacugcuauccauucgugaggugcagguacauccagcugacgagucccaaauaggacgaaacgcgc

SEQ ID NO: 10 - RNA, 81 nt, Artificial sequence
  Synthetic sequence
cggcguccuggauuccacugcuauccauucgugaggugcagguacauccagcugacgagucccaaauaggacgaaacgccg

SEQ ID NO: 11 - RNA, 69 nt, Artificial sequence
  Synthetic sequence
gcgcguccuggauuccacugcuucggcagguacauccagcugacgagucccaaauaggacgaaacgcgc

SEQ ID NO: 12 - RNA, 65 nt, Artificial sequence
  Synthetic sequence
gcgcguccuggauuccacuuucgagguacauccagcugacgagucccaaauaggacgaaacgcgc

SEQ ID NO: 13 - RNA, 63 nt, Artificial sequence
  Synthetic sequence
gcgcguccuggauuccacuucggguacauccagcugacgagucccaaauaggacgaaacgcgc

SEQ ID NO: 14 - RNA, 61 nt, Artificial sequence
  Synthetic sequence
gcgcguccuggauuccauucgguacauccagcugacgagucccaaauaggacgaaacgcgc

SEQ ID NO: 15 - RNA, 63 nt, Artificial sequence
  Synthetic sequence
gcgcguccuggauucgcauucgcguacauccagcugacgagucccaaauaggacgaaacgcgc

SEQ ID NO: 16 - RNA, 65 nt, Artificial sequence
  Synthetic sequence
gcgcguccuggauucgcgauuccgcguacauccagcugacgagucccaaauaggacgaaacgcgc

SEQ ID NO: 17 - RNA, 67 nt, Artificial sequence
  Synthetic sequence
gcgcguccuggauucgcgcauucgcgcguacauccagcugacgagucccaaauaggacgaaacgcgc

SEQ ID NO: 18 - RNA, 65 nt, Artificial sequence
  Synthetic sequence
gcgcguccuggauucgcguucgcgcguacauccagcugacgagucccaaauaggacgaaacgcgc

SEQ ID NO: 19 - RNA, 65 nt, Artificial sequence
  Synthetic sequence
gcgcguccuggauucgcggaaacgcguacauccagcugacgagucccaaauaggacgaaacgcgc

SEQ ID NO: 20 - RNA, 65 nt, Artificial sequence
  Synthetic sequence
gcgcguccuggauucgcgucaccgcguacauccagcugacgagucccaaauaggacgaaacgcgc

SEQ ID NO: 21 - RNA, 70 nt, Artificial sequence
  Synthetic sequence
gcgcguccuggauucgcgagaggaggccgcguacauccagcugacgagucccaaauaggacgaaacgcgc

SEQ ID NO: 22 - RNA, 72 nt, Artificial sequence
  Synthetic sequence
gcgcguccuggauucggccagaggaggcggccguacauccagcugacgagucccaaauaggacgaaacgcgc

SEQ ID NO: 23 - RNA, 44 nt, Artificial sequence
  Synthetic sequence
cuggauuccacugcuauccauucgugaggugcagguacauccag

SEQ ID NO: 24 - RNA, 44 nt, Artificial sequence; identical to SEQ ID NO: 23
  Synthetic sequence

SEQ ID NO: 25 - RNA, 28 nt, Artificial sequence
  Synthetic sequence
cuggauuccacuuucgagguacauccag

SEQ ID NO: 26 - RNA, 26 nt, Artificial sequence
  Synthetic sequence
cuggauuccacuucggguacauccag

SEQ ID NO: 27 - RNA, 24 nt, Artificial sequence
  Synthetic sequence
cuggauuccauucgguacauccag

SEQ ID NO: 28 - RNA, 22 nt, Artificial sequence
  Synthetic sequence
cuggauuccuucguacauccag

SEQ ID NO: 29 - RNA, 22 nt, Artificial sequence
  Synthetic sequence
cuggauuccuucuuacauccag

SEQ ID NO: 30 - RNA, 26 nt, Artificial sequence
  Synthetic sequence
cuggauucgcauucgcguacauccag

SEQ ID NO: 31 - RNA, 28 nt, Artificial sequence
  Synthetic sequence
cuggauucgcgauuccgcguacauccag

SEQ ID NO: 32 - RNA, 30 nt, Artificial sequence
  Synthetic sequence
cuggauucgcgcauucgcgcguacauccag

SEQ ID NO: 33 - RNA, 28 nt, Artificial sequence
  Synthetic sequence
cuggauucgcgauuccgcguacauccag

SEQ ID NO: 34 - RNA, 28 nt, Artificial sequence
  Synthetic sequence
cuggauucgcguucgcgcguacauccag

SEQ ID NO: 35 - RNA, 28 nt, Artificial sequence
  Synthetic sequence
cuggauucgcggaaacgcguacauccag

SEQ ID NO: 36 - RNA, 28 nt, Artificial sequence
  Synthetic sequence
cuggauucgcgucaccgcguacauccag

SEQ ID NO: 37 - RNA, 33 nt, Artificial sequence
  Synthetic sequence
cuggauucgcgagaggaggccgcguacauccag

SEQ ID NO: 38 - RNA, 35 nt, Artificial sequence
  Synthetic sequence
cuggauucggccagaggaggcggccguacauccag

SEQ ID NO: 39 - DNA, 25 bp, Artificial sequence
  Synthetic sequence
cgaatggatagcagtggaatccagg

SEQ ID NO: 40 - DNA, 25 bp, Artificial sequence
  Synthetic sequence
ctggatgtacctgcacctcacgaat

SEQ ID NO: 41 - DNA, 25 bp, Artificial sequence
  Synthetic sequence
ggactcgtcagctggatgtacctgc

SEQ ID NO: 42 - DNA, 25 bp, Artificial sequence
  Synthetic sequence
ttcgtcctatttgggactcgtcagc

SEQ ID NO: 43 - DNA, 24 bp, Artificial sequence
  Synthetic sequence
gatgtacccgaagtggaatccagg

SEQ ID NO: 44 - DNA, 25 bp, Artificial sequence
  Synthetic sequence
gtacccgaagtggaatccaggacgc

SEQ ID NO: 45 - DNA, 22 bp, Artificial sequence
  Synthetic sequence
cgtttccgcgaatccaggacgc

SEQ ID NO: 46 - DNA, 25 bp, Artificial sequence
  Synthetic sequence
gccgcctcctctggccgaatccagg

SEQ ID NO: 47 - DNA, 25 bp, Artificial sequence
  Synthetic sequence
gatagcagtggaatccaggacgcac

SEQ ID NO: 48 - DNA, 25 bp, Artificial sequence
  Synthetic sequence
gcgtcctggattccacttcgggtac

(19) United States
(12) Patent Application Publication
(10) Pub. No.: US 2023/0265422 A1
(43) Pub. Date: Aug. 24, 2023

(54) EFFICIENT RNA SWITCHES AND RELATED EXPRESSION SYSTEMS

(71) Applicant: THE SCRIPPS RESEARCH INSTITUTE, (US)

(72) Inventors: Michael Farzan, Juno Beach, FL (US); Guocai Zhong, Jupiter, FL (US); Haiming Wang, Jupiter, FL (US)

(21) Appl. No.: 17/768,896

(22) PCT Filed: Oct. 14, 2020
(86) PCT No.: PCT/US20/55495

§ 371 Date: Apr. 14, 2022

Related U.S. Application Data
(60) Provisional application No. 62/915,258, filed on Oct. 15, 2019.

(51) Int. Cl. C12N 15/113; C12N 15/63

(52) U.S. Cl. CPC C12N 15/113; C12N 15/635; C12N 2310/121; C12N 2310/128

(57) ABSTRACT

The present invention provides novel RNA switches that are based on modified hammerhead ribozymes with improved activities. Also provided are expression vectors and related expression systems for regulating transgene expression in various clinical or industrial applications.

### CROSS-REFERENCE TO RELATED APPLICATIONS

The subject patent application claims the benefit of priority to U.S. Provisional Patent Application No. 62/915,258 (filed Oct. 15, 2019). The full disclosure of the priority application is incorporated herein by reference in its entirety and for all purposes.

### STATEMENT CONCERNING GOVERNMENT SUPPORT

This invention was made with government support under Grant No. DA043912 awarded by the National Institutes of Health. The government has certain rights in the invention.

### BACKGROUND OF THE INVENTION

Systems that permit external regulation of gene expression can be applied in many scientific and medical contexts. These include investigations of protein function, and the control of expression of biologics delivered by gene therapy vectors. One approach to such regulation has been inspired by natural riboswitches, structured noncoding RNA domains which bind specific metabolites to control gene expression. Engineered riboswitches typically rely on the allosteric regulation of a self-cleaving ribozyme such as the Schistosoma mansoni hammerhead (HH) ribozyme. These HH ribozymes cleave themselves in cis with high specificity, and their enzymatic activities depend solely on their conformation, and not on any protein cofactor. RNA-based switches have two key strengths over protein-dependent transcriptional switches for gene-therapy applications. First, these switches are generally small (<200 bp), and thus can be easily incorporated into gene-therapy vectors with limited packaging capacity, for example those based on adeno-associated virus (AAV). Second, RNA switches do not require a potentially immunogenic non-self protein such as the rtTA protein for the Tet-On transcriptional activation system.

Nonetheless, most RNA-based switches suffer from a narrow regulatory range, which usually precludes their use in vivo. This narrow range can be a consequence of limited efficiency of the RNA effector domain or poor response to the exogenous control agent. For example, hammerhead ribozymes are widely-used effector domains for RNA switches. However, introduction of the most efficient of these ribozymes at the 3′ UTR of a reporter gene, an optimal location for gene regulation, affords only an 18-fold decrease in reporter expression relative to that observed with an inactive form of the same ribozyme in cell culture. This dynamic range represent an upper theoretical bound on regulation using these ribozymes and is generally unsuitable for most in vivo applications.

A strong need exists in the art for more efficient and broadly applicable polynucleotide switches for controlling gene expressions in various settings such as gene therapy. The present invention is directed to this and other unmet needs in the art.

### SUMMARY OF THE INVENTION

In one aspect, the invention provides type III hammerhead (HH) ribozymes that are modified from a type I cis-acting hammerhead ribozyme. These engineered variant HH ribozymes have the 5′ and 3′ ends of its sequence in stem III. In some embodiments, the type III variant hammerhead (HH) ribozymes of the invention are modified from a type I HH ribozyme derived from Schistosoma mansoni. In various embodiments, the type I HH ribozyme from which the type III variant ribozymes are based or engineered can be, e.g., ribozyme N107, N117 or a natural Schistosoma mansoni HH ribozyme. In one embodiment, the type III hammerhead (HH) ribozyme of the invention can contain the sequence (5′-3′) accgg ugcgu ccugga uucca cugcu aucca uucgu gaggu gcagg uacau ccagc ugacg agucc caaau aggac gaaac gcgcc ggu (SEQ ID NO:8).

In some embodiments, the type III hammerhead (HH) ribozyme of the invention can additionally include one or more modifications of the sequence of the type I HH ribozyme. These additional modifications can be, e.g., (a) optimization of stem III of the resulting type III ribozyme and/or (b) modifications of stem I and loop I to facilitate formation of the “UAC” bulge at stem I and stabilize the tertiary interactions between the “UAC” bulge and loop II. In some embodiments, the optimized stem III contains from about 4 to about 8 pair pairs, and has a calculated annealing energy of about −9 kcal/mol. In some of these embodiments, the two sequences on the opposite sides of the optimized stem III respectively contain residues (5′-3′/3′-5′) acgcgc/ugcgcg or acgccg/ugcggc. In some of these embodiments, the type III variant HH ribozyme contains the sequence (5′-3′) as shown in SEQ ID NO:9 or SEQ ID NO:10.

In some embodiments, the two sequences on the opposite sides of the modified stem I, immediately 5′ to the “UAC” bulge, are perfectly matched. In some of these embodiments, the two sequences on the opposite sides of the modified stem I, immediately 5′ to the “UAC” bulge, respectively contain residues (5′-3′/3′-5′) cc/gg, cgc/gcg, cgcg/gcgc, or cgcgc/gcgcg. In some embodiments, the altered loop I contains the sequence (5′-3′) auuc, uucg, gaaa, ucac, or agaggaggc.

Some type III variant HH ribozymes of the invention contain both an optimized stem III and a modified stem I. In some of these embodiments, the variant ribozyme contains the sequence (5′-3′) shown in any one of SEQ ID NOs:11-17.

Some type III variant HH ribozymes of the invention contain an optimized stem III, a modified stem I and an altered loop I. In some of these embodiments, the variant ribozyme contains the sequence (5′-3′) shown in any one of SEQ ID NOs:18-22.

In another aspect, the invention provides expression vectors that contain a target gene sequence that is operably fused to a type III HH ribozyme-coding sequence. In some of these vectors, the type III HH ribozyme is an engineered variant of type I cis-acting HH ribozyme N107, N117 or a natural Schistosoma mansoni HH ribozyme. In these vectors, and the HH ribozyme-coding sequence is typically inserted into the target gene at its 3′-UTR or 5′-UTR. In some embodiments, the vector further contains one or more transcriptional regulatory sequences that regulate transcription of the target gene in a mammalian cell. Some of the vectors are DNA vectors.

In various embodiments, the expression vector of the invention contains one or more additional modifications of the sequence of the type I cis-acting HH ribozyme. These additional modifications include, e.g., (a) optimization of stem III to have a calculated annealing energy of about −9 kcal/mol and/or (b) modifications of stem I and loop Ito facilitate formation of the “UAC” bulge at stem I and stabilize the tertiary interactions between the “UAC” bulge and loop II. In some vectors, the encoded HH ribozyme contains the sequence (5′-3′) shown in any one of SEQ ID NOs:9-22. In some expression vectors, the target gene encodes erythropoietin (Epo). In a related aspect, the invention provides engineered mammalian cells that harbor an expression vector described herein.

In another aspect, the invention provides methods for inducing expression of a target gene in a cell. The methods involve (a) constructing an expression vector described herein, (b) introducing the expression vector into the cell, and (c) contacting the cell with an RNase H-independent antisense oligonucleotide that is complementary to the HH ribozyme-coding sequence in the expression vector. This will allow induction of expression of the target gene in the cell. In some embodiments, the RNase H-independent antisense oligonucleotide used in the methods is a morpholino oligonucleotide. In some embodiments, the employed host cell for the expression induction is a mammalian cell. In some embodiments, the expression vector is an adeno-associated virus (AAV) vector.

In some methods of the invention, the HH ribozyme encoded by the vector contains one or more additional modifications of the sequence of the type I cis-acting HH ribozyme. The additional modifications can be, e.g., (a) optimization of stem III to have a calculated annealing energy of about −9 kcal/mol and/or (b) modifications of stem I and loop I to facilitate formation of the “UAC” bulge at stem I and stabilize the tertiary interactions between the “UAC” bulge and loop II. In some methods, the HH ribozyme encoded by the vector contains the sequence (5′-3′) shown in any one of SEQ ID NOs:9-22. In some methods, the target gene for expression induction encodes erythropoietin (Epo). In some embodiments, the employed RNase H-independent antisense oligonucleotide is a modified morpholino. In some of these methods, the modified morpholino is an octa-guanidine dendrimer-coupled morpholino. In various embodiments, the employed RNase H-independent antisense oligonucleotide contains the oligonucleotide sequence as shown in any one of SEQ ID NOs:38-46.

In some methods of the invention, the host cell for inducing expression of a target gene is present in a subject in need of the polypeptide encoded by the target gene. In some methods, the cell is obtained from the subject prior to introduction of the expression vector into the cell. Some of the methods additional include reintroducing the cell into the subject after introducing the expression vector into the cell. In various methods, the RNase H-independent antisense oligonucleotide can be administered to the subject prior to, simultaneous with, or subsequent to reintroducing the cell into the subject.

A further understanding of the nature and advantages of the present invention may be realized by reference to the remaining portions of the specification and claims.

### DESCRIPTION OF THE DRAWINGS

FIG. 1 illustrates schematically development of a class of highly efficient hammerhead ribozymes (HHR). (a) A diagram representing HHR-mediated inactivation of gene expression. (b) A panel of engineered HHR variants were tested in a reporter assay in which an expression vector encoding Gaussia luciferase (Gluc) and Cypridina luciferase (Cluc), each driven by an independent promoter, was used. A catalytically active ribozyme variant was placed to the 3′ UTR of the Gluc gene, and compared with a catalytically inactive form of the same ribozyme. Reporter plasmids were transfected into 293T cells, and the functional ribozyme's activity was calculated as fold inhibition in the Gluc expression relative to the inactive ribozyme control. Cluc activity was simultaneously monitored to control for dose and transfection efficiencies. As shown, the catalytic activity of the previously described Schistosoma mansoni HHR variant, N107, was improved from its original 18-fold to 1,200-fold (reflected in the bars) through a series of modifications, as represented above the figure. These include conversion of the type I HHR N107 to a type III HHR, optimization of stem III of the resulting type III ribozymes, modification of stem I to stabilize the tertiary interactions essential for ribozyme function, and alteration of loop I to facilitate hairpin formation. (c) Sequences and secondary structures of a natural Schistosoma mansoni HHR (SEQ ID NO:1), the N107 ribozyme (SEQ ID NO:4), and several milestone type III ribozyme variants characterized in panel b, including T3H1 (SEQ ID NO:8), T3H16 (SEQ ID NO:9), T3H38 (SEQ ID NO:13), and T3H48 (SEQ ID NO:19). Nucleotide differences among the ribozyme variants are indicated in black. Further details are provided in FIG. 5. Data points in panel b represent mean±s.e.m of three or four independent experiments.

FIG. 2 shows efficient regulation of gene expression using optimized type III ribozymes. (a) A diagram showing how an antisense oligonucleotide can inactivate a ribozyme to rescue protein expression. (b) The sequence (SEQ ID NO:9) and secondary structure of T3H16 ribozyme and the target regions complementary to the panel of phosphorodiamidate morpholino oligomers characterized in panels c through e. (c) 293T or Huh7 cells transfected with catalytically active or inactive T3H16 ribozyme-regulated reporter plasmids were treated with 10 μM of the indicated morpholino variants (M2 through M6) or an identical length control morpholino (NC). A functional morpholino's activity was calculated as fold induction in the Gluc reporter expression relative to the Gluc expression from the NC control morpholino-treated cells. As shown, the M3 morpholino induced the highest reporter activity, 13.6-fold and 3.6-fold in 293T cells and Huh7 cells, respectively. (d) Experiments similar to those in panel c except that the indicated cells were treated with an octa-guanidine dendrimer-coupled form of the M3 oligo, v-M3, and a similarly coupled control oligo, v-NC. Octa-guanidine dendrimer coupling improved the induction activity of M3 oligo by more than 10-fold relative to the unmodified morpholino shown in panel c. (e) v-M3 morpholino-mediated induction of Gluc was compared using reporter plasmids with the T3H16 ribozyme placed at 3′ UTR or 5′ UTR. v-M3R is randomized from the v-M3 sequence. Note that induction is significantly greater when the ribozyme is placed at 3′ UTR. (f) The sequences and secondary structures of three ribozyme variants, which have greater catalytic activity than T3H16 (T3H38 (SEQ ID NO:13) and T3H48 (SEQ ID NO:19)) or larger stem-I loop for morpholino targeting (T3H52 (SEQ ID NO:22)), and the complementary target regions of the indicated morpholinos. (g) Octa-guanidine dendrimer-coupled forms of morpholinos M3 and M7-M10 (v-M3, v-M7 through v-M10) were tested in 293T cells for induction of Gluc expression from the corresponding ribozyme variant-controlled reporter plasmid. Numbers above the figure indicate the fold induction mediated by each morpholino. Morpholino v-M8 showed the highest specific induction of Gluc expression from a T3H38 ribozyme-regulated reporter plasmid (319-fold). This morpholino-ribozyme pair was therefore chosen to be tested in the subsequent animal studies. Data shown are representative of two or three independent experiments with similar results, and data points represent mean±s.d. of three biological replicates.

FIG. 3 shows in vivo induction of an adeno-associated virus (AAV) reporter transgene. (a) A diagram representing the AAV vector and the experimental design used in the animal studies of panels b through e. (b) Six-week old male BALB/c mice were injected in the left gastrocnemius muscle with 1×10¹⁰ genome copies (GC) of AAV particles encapsidating a firefly luciferase (Fluc) gene with an active or inactive T3H38 ribozyme at its 3′ UTR. Two weeks post AAV inoculation, mice were intramuscularly (i.m.) injected at the same site with 0.5 or 2.5 mg/kg of with a control (v-NC) or the v-M8 morpholino. Luciferase expression was measured at days 0 and 2 post-morpholino injection using the Xenogen IVIS In-Vivo Imager. (c) Quantitation of luciferase expression shown in panel b. (d) An experiment similar to that shown in panel b except that both the left and right gastrocnemius muscles of 8-week old female BALB/c mice were injected with 5×10⁹ GC of T3H38-regulated AAV-Fluc. One group of mice then received an i.m. injection of 0.5 mg/kg v-M8 morpholino to left gastrocnemius muscle and PBS to the right gastrocnemius muscle. The other group of mice received 12.5 mg/kg v-M8 morpholino via tail vein injection. Quantification of in vivo bioluminescence for the indicated time points is shown. Data points represent mean±s.d. of five mice per group. Representative bioluminescent images for basal expression and induced peak expression are shown. (e) An experiment similar to that shown in panels b through d except that two randomly assigned groups of 8-week old female BALB/c mice were injected multiple times with a control (v-NC) or v-M8 morpholino as indicated above the arrows. In vivo bioluminescent data for weeks 2 to 43 post AAV inoculation are shown. Numbers beside each peak indicate fold-induction over background measured before morpholino administration. Images associated with this figure are shown in FIG. 8a. Data points represent mean±s.d. of four mice per group.

FIG. 4 shows in vivo regulation of an erythropoietin (Epo) transgene. (a) A diagram representing the AAV vector and the experimental design used in the animal studies of panels b through h. (b) Eight-week old female BALB/c mice were i.m. injected with 1×10¹⁰ GC of AAV particles carrying a mouse erythropoietin (Epo) gene and an catalytically active (upper right panel) or inactive (lower left panel) T3H38 ribozyme at 3′ UTR of the Epo gene. Mice injected with active ribozyme-regulated AAV were further treated with 0.5 mg/kg of the v-M8 morpholino on day 12 post AAV inoculation as indicated. Mice received no treatment (upper left panel) and a group of mice intraperitoneally (i.p.) injected with 3 μg recombinant mouse Epo protein (lower right panel) were used as controls. Tail-vein blood samples were collected at the indicated time points, and hematocrit counts (gray lines with squares) and plasma Epo protein concentrations (black lines with circles) were measured. Each line represents values obtained from a single mouse. (c-d) Experiments similar to panel b except that the mice were injected with 5×10⁹ GC of AAV particles carrying active T3H38 ribozyme-regulated mouse Epo gene and then treated with the indicated doses (0, 0.1, 0.5, or 2.5 mg/kg) of the v-M8 morpholino. Arrows indicate the time of morpholino injection. Plasma Epo protein concentrations (c) and hematocrit counts (d) were measured using tail-vein blood samples. All differences among sets of mice treated with different morpholino concentrations are significant (paired sample Student's t-test, P<0.01 for panel c, and P<0.05 for panel d) except for hematocrit values at 0.5 and 2.5 mg/kg. (e-f) Experiments similar to panel b except that the mice were injected with 2×10⁹ GC of AAV particles and then treated with 0.5 mg/kg of the v-M8 morpholino. Mice receiving morpholino had significantly greater (paired sample Student's t-test, P<0.01) Epo and hematocrit values than those receiving PBS alone. (g) Morpholino-induced peak Epo expression data from panel c are plotted by morpholino dose. Endogenous Epo concentrations (150 pg/ml) have been subtracted to determine fold induction of AAV-expressed Epo. The half-life of Epo induction for each dose is indicated in parentheses. (h) Morpholino-induced peak Epo expression data from panels b, c, and e are plotted by AAV titers. Only induction values from mice treated with 0 or 0.5 mg/kg morpholino are shown. Again endogenous Epo concentrations have been subtracted, and the half-life of Epo induction and induction fold are indicated for each AAV dose. Data points in panels c-h represent mean±s.d. for three mice per group.

FIG. 5 shows that converting type I hammerhead (HH) ribozymes to type III significantly improved ribozyme activity (from its original 18-fold to 130-fold). (a) Sequences and secondary structures of three previously published type I hammerhead ribozyme variants (N79 (SEQ ID NO:2), N117 (SEQ ID NO:3), and N107 (SEQ ID NO:4)) and a stem I variant of N107 (N107-v1 (SEQ ID NO:5)) are shown. Watson-Crick base-pairing interactions are indicated as non-bold lines. Other hydrogen-bonding interactions are represented as thin black lines with annotations according to the following key: open circle next to open square represents a Watson-Crick/Hoogsteen interaction; open square next to open triangle, a Hoogsteen/sugar edge interaction; line with T termini, nonadjacent base stacking; circle, a Wobble base pair. Thick solid or dashed non-bold lines indicate backbone continuity where the sequence has been separated for diagrammatic clarity. Thick black lines indicate flexible linker sequences introduced to isolate the ribozyme and help stem-I folding. Arrows indicate a single A to G mutation that inactivates the ribozyme. Nucleotide differences among the variants are indicated in black. Numbers in the parentheses are the fold inhibition activities of each variant when they are placed at 3′ UTR of a Gaussia luciferase (Gluc) reporter gene and tested in 293T cells using the reporter inhibition assay described in FIG. 1. (b) A potential explanation for the differences in reaction rates between a type I and type III HEIR is diagramed. The enzyme strand of the HHR is indicated in gray, and the substrate strand is indicated in black. A cross indicates the HHR cleavage site. The short substrate strand that dissociates after cleavage is referred as the leaving strand. The base-pairing interactions holding the long leaving strand of stem I and the tertiary interactions between this strand and loop II may slow the disassembly of the cleaved type I ribozyme and facilitate re-ligation of the cleaved substrate strand. In contrast, a cleaved type III HHR may disassemble more quickly with less re-ligation, due to its shorter leaving strand and the absence of tertiary interactions between its leaving strand and the remainder of the ribozyme. (c) Sequence and secondary structure of T3H1 ribozyme (SEQ ID NO:8), a type III HHR converted in this study from the previously described HHR ribozyme, N107. Nucleotide differences from N107 ribozyme are indicated in black. (d-e) N107-v1 and T3H1 were compared for their activities in a reporter inhibition assay, where a dual-reporter expression vector encoding a Gaussia luciferase (Gluc) and a Cypridina luciferase (Cluc), expressed from two independent promoters, was used. A catalytically active ribozyme variant was placed to the 3′ UTR (d) or 5′ UTR (e) of the Gluc gene, and compared with a catalytically inactive form of the ribozyme placed to the same sites. Reporter plasmids were transiently transfected into the indicated cell lines, and the ribozyme's activity was calculated as fold inhibition in the Gluc expression relative to the inactive ribozyme control. A Cluc reporter was used as an internal control of Gluc expression. Consistent with the hypothesis represented in (b), type III ribozyme T3H1 outperformed type I ribozyme N107-v1 when placed at the 3′ UTR. Interestingly, no significant differences were observed when T3H1 and N107 were placed at 5′ UTR, perhaps because cellular RNA helicases promote disassembly of both ribozymes. Thus ribozyme activity at the 5′UTR might indicate an initial cleavage rate independent of the rate of disassembly. Data shown are representative of two or three independent experiments with similar results, and data points represent mean±s.d. of three or four biological replicates.

FIG. 6 shows that optimizing stem III sequences to balance cleavage and disassembly rates further improved ribozyme activity (from 130- to 300-fold). (a) A diagram representing a proposed difference between enzymatic reactions of type III HHRs with different stem-III annealing energies. A more stable stem III facilitates cleavage but slows disassembly, whereas a less stable stem III works in the opposite direction. Thus an optimal stem III sequence that balances these processes affords the maximum inhibition of gene expression. The enzyme strand of the ribozyme is indicated in gray, and the substrate strand is indicated in black. A cross indicates the cleavage site. (b-c) Stem-III sequences (b) and fold inhibition activities (c) of a panel of T3H1 variants, which differ from T3H1 only at its stem III region. Consistent with the hypothesis diagrammed in (a), ribozyme activity at 3′ UTR increases as the stem III shortens from 8-bp to 5-bp, indicating that accelerated disassembly overcomes any loss of intrinsic catalytic activity. (d) The ribozyme variants in (b) were placed at 5′ UTR, where the disassembly of a cleaved ribozyme may largely depend on the cellular RNA helicase activity. No significant change in ribozyme activity was observed when stem III was shortened from 8-bp to 6-bp. However, ribozyme cleavage activity decreased dramatically when the stem III was shortened from 6-bp to 4-bp, consistent with a loss of intrinsic activity with a less stable stem III. (e) Stem-III sequences of a panel of ribozyme variants, modified from those in (b) to have maximal numbers of potential inter-strand purine-base stackings in stem III. (f) The ribozyme variants in (e) were placed at 3′ UTR and compared with the ribozymes in (b) for their fold inhibition activities in 293T cells. Note that a 6-bp stem III is optimal for both ribozyme panels, and that the 6-bp variant T3H16 exhibited the highest fold inhibition. (g-h) Stem-III sequences (g) and fold inhibition activities in 293T cells (h) of a panel of T3H16 variants which only differ in the number of potential inter-strand purine-base stackings. Ribozymes T3H16 and T3H29 showed the highest fold inhibition activity, ˜300-fold. (i) The fold inhibition activities of the stem-III variants were plotted against their calculated stem-III annealing energies. Ribozyme activities show a negative skewed distribution, and both T3H16 and T3H29 have annealing energies of −9.15±0.05 kcal/mol. Data shown are representative of two or three independent experiments with similar results, and data points represent mean±s.d. of three or four biological replicates.

FIG. 7 shows that stabilizing tertiary interactions between stem I and loop II further improved ribozyme activity (from 300-fold to 1200-fold). (a) The secondary structure of a crystalized Schistosoma mansoni HHR (SEQ ID NO:1) and the tertiary structure of a loop II-interacting bulge in its stem I are shown (PDB: 3ZD5). The extensive tertiary interactions between this bulge and loop II are essential for the function of an HHR in mammalian cells. Based on the crystal structure, another smaller bulge above the loop II-interacting bulge may destabilize these tertiary interactions. We therefore sought to stabilize this loop II-interacting bulge through stem-I changes. (b) Stem-I sequences of a panel of T3H16 variants (SEQID NOs:24-32), which differ from T3H16 Stem I (SEQ ID NO:23) only in stem I. Loop II-interacting bulges are shown in gray. Numbers in the parentheses indicate fold-inhibition activities of the corresponding ribozymes in 293T cells. (c-d) Sequences (c) and fold inhibition activities in 293T cells (d) of a panel of ribozyme variants carrying nucleotide mutations (bold font) at the bulge I/loop II region. T3H54 and T3H56 are T3H44 variants bearing single-nucleotide changes designed to impair bulge I/loop II tertiary interactions, and serve here as controls. Similarly, T3H57 is a variant of T3H41 with a single, destabilizing nucleotide change. (e) The stem I sequences of a panel of T3H44 variants (SEQ ID NOs:33-38) differing from T3H44 only in the regions indicated in bold font. Numbers in the parentheses indicate fold inhibition activities of the corresponding ribozymes in 293T cells. (f) Fold-inhibition activities in 293T cells of the indicated hammerhead ribozyme variants at the 5′ UTR or 3′ UTR of the Gluc reporter gene. (g-h) Sequences (g) and fold-inhibition activities in 293T cells (h) of N107 (SEQ ID NO:4), N117, three N107-derived type-III hammerhead ribozyme variants (T3H16 (SEQ ID NO:9), T3H36 (SEQ ID NO:11), T3H37 (SEQ ID NO:12)), and three N117-derived type-III hammerhead ribozyme variants (T3H16×117, T3H36×117, T3H37×117). (i-j) Sequences (i) and fold inhibition activities in 293T cells (j) of four type III hammerhead ribozyme variants (from left to right, SEQ ID NOs:9, 19, 6, and 7). sTRSV-H1 (SEQ ID NO:6) is a natural type III hammerhead ribozyme from satellite tobacco ringspot viral genome. sTRSV-H2 (SEQ ID NO:7) is a ribozyme variant identical to sTRSV-H1 except that the stem III sequence is replaced with the sequence from T3H16 (SEQ ID NO:9). Calculated stem-III annealing energy values for sTRSV-H1 and sTRSV-H2 are shown in parentheses. Data shown are representative of two or three independent experiments with similar results, and data points in panels d, f, and h represent mean±s.d. of three or four biological replicates

FIG. 8 shows cell-type dependent activities of ribozyme variants and the v-M8 morpholino. (a) Fold-inhibition of reporter expression with the indicated ribozyme variants in five different cell lines. Note that the rank order of ribozyme regulation remains consistent across cell lines. (b) T3H38 ribozyme and v-M8 morpholino were tested in the five indicated cell lines for induction of Gluc expression. Gluc expression levels were normalized against the expression from a control reporter construct that carries no ribozyme element. v-M8 morpholino-mediated fold induction of active T3H38-regulated Gluc expression is shown for each cell line. Data points represent mean±s.e.m of two or three independent experiments, each with three or four biological replicates.

FIG. 9 shows in vivo regulation of adeno-associated virus (AAV)-delivered reporter expression in mouse muscle. (a) Luciferase expression in the same mice shown in panel b of FIG. 3 was monitored over a 5-week period. Data points represent mean±s.d. of three mice per group. (b) Bioluminescence images of mice used to generate the data of FIG. 3d. (c) Eight-week old female BALB/c mice received bilateral intramuscular (i.m.) injections of T3H38-regulated AAV-Fluc at 5×10⁹ GC/leg. Mice then received daily intravenous (i.v.) injections of PBS or 12.5 mg/kg v-M8 morpholino from day 7 to day 10 post AAV injection. In vivo bioluminescent imaging was performed at the indicated time points using the Xenogen IVIS In-Vivo Imager. (d) In vivo bioluminescent quantification data for the mice images in panel c. Data points represent mean±s.d. of five mice per group.

FIG. 10 shows off-target activity of the v-M8 morpholino. (a) Sequences of v-M8 morpholino (SEQ ID NO:44) and its complement, v-M8′ (SEQ ID NO:47). (b) BLAST analysis was performed on the human transcriptome to identify transcript sequences similar to the v-M8′. Identifiers and sequence information of 11 potential off-target transcripts that are PCR-detectable in 293T cellular RNAs are listed. (c) RT-qPCR analysis for these 11 potential off-target transcripts using total RNA samples from 293T cells receiving no morpholino treatment, or treated with 10 μM of a control morpholino (v-M3R) or the v-M8 morpholino. Relative quantification of each potential off-target transcript was performed using GAPDH as an internal control. The abundance of each transcript was normalized to the abundance of the same transcript detected in the “No morpholino” control samples. v-M8 morpholino treatment significantly increased abundance of the SEMA6A-AS2, GRSF1 transcript variant X1, SMIM10L1, and IKZF2 transcript variant X23 (two-sample Student's t-test, P<0.01). Data points represent mean±s.e.m of two independent experiments, each with two biological replicates.

FIG. 11 shows in vivo regulation of AAV-delivered reporter expression in mouse muscle and liver. (a) Bioluminescence images of the two groups of mice used to generate data of FIG. 3e. Group-1 mice received i.m. injections of 0.5 mg/kg v-NC, 0.5 mg/kg v-M8, 0.5 mg/kg v-M8, and 2.5 mg/kg v-M8 at weeks 2, 7, 15, and 38 post AAV injection, respectively. Group-2 mice received i.m. injections of 0.5, 0.5, and 2.5 mg/kg v-M8 at weeks 2, 7, and 38 post AAV injection, respectively. In vivo bioluminescent images for the indicated time points are shown. (b) The same mice used in panel a were i.m. injected again with 2.5 mg/kg v-M8 morpholino at day 600 post AAV injection. In vivo bioluminescent imaging was performed at the indicated time points. (c) Six- to eight-week old BALB/c or C57BL/6J mice were injected with 1×10¹⁰ GC of catalytically active or inactive T3H38-regulated AAV-Fluc. I.m. injection of AAV1 particles was performed for muscle transduction, while i.v. injection of AAV8 particles was performed for liver transduction. In vivo bioluminescent imaging was performed at week 1 post AAV injection. Each bar indicates the mean and range of the luciferase signals of each mouse group. The numbers of mice used in each group are indicated next to each bar. (d-e) Eight-week old female BALB/c mice were i.v. injected with 1×10¹⁰ GC of AAV particles expressing firefly luciferase regulated by an active T3H38 ribozyme. Seventeen days later, mice were injected i.v. daily with PBS or 12.5 mg/kg of v-M8 morpholino for four days. Luciferase expression at the indicated time points was measured using the Lago X optical imaging system. Image data (d) and quantification of bioluminescence (e) are shown. Data points represent mean±s.d. of four mice per group.

FIG. 12 shows that intravenous injections of high doses of v-M8 morpholino do not affect mouse body weight. (a) Nineteen-week old female BALB/c mice were i.v. injected with v-M8 morpholino at 0, 2.5, 5.0, or 10.0 mg/kg/day for two days. Mouse body weight was measured at the indicated time points. Arrows indicate v-M8 injections. Each line represents body weight data collected from a single mouse. No significant change on body weight was observed for mice treated with different doses of v-M8 morpholino (paired-sample Student's t-test, P>0.05). (b) Experiments similar to (a) except that eight-week old female BALB/c mice were i.v. injected with v-M8 morpholino at 0 or 12.5 mg/kg/day for four days. No significant change on body weight was observed for the two mouse groups (paired-sample Student's t-test, P>0.05).

### DETAILED DESCRIPTION OF THE INVENTION

### I. Overview

Gene therapy has begun to provide impressive therapeutic benefits and potential cures for a range of human diseases, including inherited genetic diseases, cancers, and infectious diseases. Most of these applications use an AAV vector to express a therapeutic protein, an approach favored because the safety of these vectors and their relatively lower immunogenicity. RNA-based switches, with their small size and lack of dependence on exogenous proteins, make ideal regulators of AAV transgenes, but their narrow regulatory ranges preclude their in vivo use. A single exception to this is an on-switch system built from an engineered HH ribozyme similar to the well-known HH ribozyme N107 (Yen Nature 431, 471-476, 2004; and Yan et al., RNA 12, 797-806, 2006). Nonetheless, regulation of this system is mediated by a compound that disrupts ribozyme function by directly incorporating a nucleoside analogue into the transgene mRNA, and thus would not be suitable for clinical applications. In summary, widespread use of gene therapy technologies is limited by the lack of useable genetic switches for controlling transgene expression. Transcriptional regulatory switches are efficient but require parallel expression of exogenous proteins, constraining transgene size and promoting immune clearance of transgene-expressing cells.

The present invention provides a novel class of RNA-based expression switches (ribozyme variants) and their uses in various therapeutic settings, e.g., gene therapies. The invention is derived in part from the studies undertaken by the inventors to engineer a class of ribozymes that are highly efficient in cis-cleaving mammalian mRNAs and can be regulated by a steric-blocking antisense oligonucleotide. The ribozymes developed by the inventors make feasible in vivo regulation of an adeno-associated virus (AAV)-delivered transgene, allowing dose-dependent control of protein expression up to 223-fold over at least 43 weeks. As detailed herein, the present invention dramatically increased the dynamic range of a HH ribozyme and combined it with an optimized morpholino to develop an on-switch system efficient enough for in vivo use. Specifically, it was observed in mice an approximately 200-fold induction of an AAV-expressed biologic, comparable to regulation mediated by exogenous transcription factors. Moreover, the dose of AAV vectors used here, 5×10⁹ genome copies per mouse, is 5 to 40 fold lower than what was used to achieve similar expression with transcriptional regulatory switches, highlighting both safety and cost-effectiveness of the system developed here. Importantly, the exemplified oligonucleotides for regulating the ribozymes of the invention, morpholinos, have the potential to be safe in humans. Indeed, a morpholino has been approved for human use for doses up to 50 mg/kg weekly infused systemically. See, e.g., Stein C. A. & Castanotto D., FDA-Approved Oligonucleotide Therapies in 2017. Mol. Ther. 25, 1069-1075, 2017. The inventors showed that morpholino concentration of 0.1 mg/kg administered locally can induce physiological levels of Epo, indicating that this inducible system is safe enough for human use.

The small, modular, and efficient RNA switches described herein can improve the efficacy and broaden the use of a range of gene therapies. As exemplification to highlight the potential of these on-switches in gene therapy for anemia of chronic kidney disease, the inventors demonstrated regulated expression of physiological levels of erythropoietin with a well-tolerated dose of the morpholino inducer oligonucleotide. Epo-deficient anemia contributes substantially to morbidity and mortality among the chronic kidney disease patients, and recombinant Epo protein is the standard of care for this condition. However, elevated risk of cardiovascular complications and death are thought associated with the high Cₘₐₓ of plasma Epo after administration of recombinant protein. Due to its short half-life, the Cₘₐₓ of recombinant Epo necessary to maintain therapeutic levels typically requires initial concentrations 6 to 60-fold over physiological Epo concentrations. With the RNA switches of the present invention, it was found that a single administration of morpholino at a well-tolerated dose could induce physiological levels of Epo with an induction half-life ˜40-times that of passively administered Epo proteins.

In addition, there are also a number of other advantages in using on-switch systems based on the ribozyme variants and morpholino oligonucleotide inducers described herein to control transgene expression in gene therapies. First, a tightly regulated AAV-based transgene expression system such as the Epo expression system demonstrated here could circumvent safety concerns associated with current standard of care for anemia of chronic kidney disease. In addition, the absence of exogenous regulatory proteins, and the ability to delay transgene expression to well after AAV-induced innate responses subside, can prevent the emergence of anti-transgene antibodies observed with other AAV-based systems (see, e.g., Chenuaud, Blood 103, 3303-3304, 2004; and Gao et al., Blood 103, 3300-3302, 2004). Moreover, the expression of Epo from human tissue can also obviate immune responses observed with Epo manufactured in non-human cell lines (see, e.g., Casadevall, N. Engl. J. Med. 346, 469-475, 2002; and Bunn, N. Engl. J. Med. 346, 522-523, 2002).

It is noted that this invention is not limited to using octa-guanidine dendrimer conjugated morpholino for in vivo regulation. Rather, one can improve tissue-targeting specificity and safety by changing the delivery chemistry for morpholino, such as replacing octa-guanidine dendrimer conjugation with N-acetylgalactosamine conjugation. The latter is now widely used in clinical trials for liver-specific delivery of oligonucleotides. See, e.g., Huang Y., Mol. Ther. Nucleic Acids. 6:116-132, 2017. In addition, morpholino is a class of steric-blocking antisense oligonucleotides that do not trigger RNase H cleavage of the complementary RNAs. Thus, one can replace morpholino with a completely different steric-blocking antisense chemistry, such as locked nucleic acid (LNA)/DNA mixmer (see, e.g., Hagedorn et al., Drug Discov. Today 23:101-114, 2018) or 2′-O-methoxyethyl phosphorothioate-modified antisense oligonucleotide (see, e.g., Wan and Dreyfuss. Cell. 2017. 170:5). Unless otherwise specified, this invention is not limited to the particular methodology, protocols, and reagents described as these may vary. Unless otherwise indicated, the practice of the present invention employs conventional techniques of molecular biology (including recombinant techniques), microbiology, cell biology, biochemistry and immunology, which are within the skill of the art. Such techniques are explained fully in the literature. For example, exemplary methods are described in the following references, Sambrook et al., Molecular Cloning: A Laboratory Manual, Cold Spring Harbor Press (3^{rd} ed., 2001); Brent et al., Current Protocols in Molecular Biology, John Wiley & Sons, Inc. (ringbou ed., 2003); Freshney, Culture of Animal Cells: A Manual of Basic Technique, Wiley-Liss, Inc. (4^{th} ed., 2000); and Weissbach & Weissbach, Methods for Plant Molecular Biology, Academic Press, NY, Section VIII, pp. 42 1-463, 1988. In addition, the following sections provide more detailed guidance for practicing the invention.

### II. Definitions

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1^{st} ed., 1992); Oxford Dictionary of Biochemistry and Molecular Biology, Smith et al. (Eds.), Oxford University Press (revised ed., 2000); Encyclopaedic Dictionary of Chemistry, Kumar (Ed.), Anmol Publications Pvt. Ltd. (2002); Dictionary of Microbiology and Molecular Biology, Singleton et al. (Eds.), John Wiley & Sons (3^{rd} ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000). Further clarifications of some of these terms as they apply specifically to this invention are provided herein.

As used herein, the singular forms “a”, “an”, and “the” include plural reference unless the context clearly dictates otherwise. Thus, for example, reference to “a cell” includes a plurality of such cells, reference to “a protein” includes one or more proteins and equivalents thereof known to those skilled in the art, and so forth.

As used herein, base stacking refers to interactions between two purine residues located on non-pairing but neighboring or adjacent positions (e.g., one residue apart) in a paired region of an RNA or ribozyme molecule. The interactions are due to dispersion attraction, short-range exchange repulsion, and electrostatic interactions which also contribute to stability. Base stacking effects are especially important in the secondary structure and tertiary structure of RNA. For example, RNA stem-loop structures are stabilized by base stacking in the loop region. In the rational design of HH ribozyme variants of the invention, one purine base at position N on one strand and another purine base at position N+1 or N−1 on the other strand are considered to have a potential inter-strand base-stacking interaction.

A hammerhead (HH) ribozyme contains a conserved core, three stems that extend from the core, referred to herein as stem I, stem II, and stem III, and at least one loop, which is located on the opposite end of a stems from the core. HH ribozymes can be classified into three types based on which helix the 5′ and 3′ ends are found in. If the 5′ and 3′ ends of the sequence contribute to stem I then it is a type I hammerhead ribozyme, to stem II is a type II and to stem III then it is a type III HH ribozyme. Of the three possible topological types, type I ribozymes can be found in the genomes of prokaryotes, eukaryotes and RNA plant pathogens, whereas type II ribozymes have been only described in prokaryotes, and type III ribozymes are mostly found in plants, plant pathogens and prokaryotes. As used herein, a “cis-cleaving HH ribozyme” is a HH ribozyme that, prior to cleavage, is comprised of a single polynucleotide. A cis-cleaving HH ribozyme is capable of cleaving itself. Typically, cis-acting HH ribozymes contain two loops, one located at the end of stem I or III and is referred to as loop I or III, and the other located at the end of stem II and is referred to as loop II. Trans-acting HH ribozymes contain one loop, e.g., at the end of stem II, and is referred to as loop II.

As used herein, complementary refers to a nucleotide or nucleotide sequence that hybridizes to a given nucleotide or nucleotide sequence. For instance, for DNA, the nucleotide A is complementary to T and vice versa, and the nucleotide C is complementary to G and vice versa. For instance, in RNA, the nucleotide A is complementary to the nucleotide U and vice versa, and the nucleotide C is complementary to the nucleotide G and vice versa. Complementary nucleotides include those that undergo Watson and Crick base pairing and those that base pair in alternative modes (non-Watson and Crick base pairing). For instance, as used herein for RNA, the nucleotide G is complementary to the nucleotide U and vice versa, and the nucleotide A is complementary to the nucleotide G and vice versa. Therefore, in an RNA molecule, the complementary base pairs are A and U, G and C, G and U, and A and G. Other combinations, e.g., A and C or C and U, are considered to be non-complementary base pairs.

A complementary sequence is comprised of individual nucleotides that are complementary to the individual nucleotides of a given sequence, where the complementary nucleotides are ordered such that they will pair sequentially with the nucleotides of the given sequence. Such a complementary sequence is said to be the “complement” of the given sequence. For example, complements of the given sequence, 5′-acuaguc-3′, include 3′-ugaucag-5′ and 3′-uggucgg-5′, among others. In the latter sequence, the third and sixth base pairs are both non-Watson and Crick G/U complementary base pairs.

Dynamic range (or regulation range) of a ribozyme or an RNA switch refers to the extent of expression regulation afforded by the ribozyme or RNA switch on a target sequence that is placed under the control of (i.e., operably linked to) the RNA switch. Specifically, it refers to the ratio of expression level when the switch is off (e.g., when the switch is not activated) over expression level when the switch is on.

Steric-blocking antisense oligonucleotides are molecules that bind to complementary target RNA but do not trigger RNase H-mediated degradation. Morpholino, locked nucleic acid (LNA)/DNA mixmer (see, e.g., Hagedorn et al., Drug Discov. Today 23:101-114, 2018), and 2′-O-methoxyethyl phosphorothioate-modified antisense oligonucleotide (see, e.g., Wan and Dreyfuss, Cell 170:5, 2017) are all steric-blocking antisense. Morpholinos, also known as Morpholino oligomers and as phosphorodiamidate Morpholino oligomers (PMO), are a type of oligomer molecule (colloquially, an oligo) used in molecular biology to modify gene expression. Its molecular structure has DNA bases attached to a backbone of methylenemorpholine rings linked through phosphorodiamidate groups. Morpholinos are synthetic molecules that are the product of a redesign of natural nucleic acid structure. Usually 25 bases in length, they bind to complementary sequences of RNA or single-stranded DNA by standard nucleic acid base-pairing. Morpholinos do not trigger the degradation of their target RNA molecules, unlike many antisense structural types (e.g., phosphorothioates, siRNA). Instead, Morpholinos act by “steric blocking”, binding to a target sequence within an RNA, inhibiting molecules that might otherwise interact with the RNA. Morpholinos are used as research tools for reverse genetics by knocking down gene function.

Ribozymes (ribonucleic acid enzymes) are RNA molecules that are capable of catalyzing specific biochemical reactions, similar to the action of protein enzymes. The most common activities of natural or in vitro-evolved ribozymes are the cleavage or ligation of RNA and DNA and peptide bond formation. Examples of ribozymes include the HH ribozyme, the VS ribozyme, Leadzyme and the hairpin ribozyme. Ribozyme activity can be regulated by complementary phosphorodiamidate morpholino oligomers.

Ribozymes typically contains both stems and loops. As used herein, a stem is a nucleic acid motif that extends from a ribozyme core, at least a portion of which is double-stranded. Often, there is a loop at the opposite end of the stem from the ribozyme core, and this loop connects the two strands of the double-stranded stem. A stem may contain 2 to 20 complementary base pairs, and often contains 3, 4, 5, 6, 7, 8, or 9 complementary base pairs. Stems are usually numbered according to where they extend from the core sequence. For example, a HH ribozyme can contain three stems, which are referred to as stem I, stem II, and stem III. For a given stem of the ribozyme, 100% of the nucleotides may be part of a complementary base pair. Alternatively, at least 50%, 60%, 70%, 80%, 90%, 95%, or 99% of the nucleotides in a stem are part of a complementary base pair. The remaining base pairs may be mismatched, non-complementary base pairs, or may be part of a bulge. As used herein, a loop is a sequence of nucleotides that is not paired with another strand and is located at the distal end of a stem that is opposite the core. A loop may contain between 1 to 20 nucleotides in length, more typically between 3 and 8 nucleotides in length. The loop is numbered according to the stem to which it is attached. Therefore, loop I is located at the end of stem I opposite the core, loop II is located at the end of stem II opposite the core, and loop III is located at the end of stem III opposite the core.

A “host cell” refers to a living cell into which a heterologous polynucleotide sequence is to be or has been introduced. The living cell includes both a cultured cell and a cell within a living organism. Means for introducing the heterologous polynucleotide sequence into the cell are well known, e.g., transfection, electroporation, calcium phosphate precipitation, microinjection, transformation, viral infection, and/or the like. Often, the heterologous polynucleotide sequence to be introduced into the cell is a replicable expression vector or cloning vector. In some embodiments, host cells can be engineered to incorporate a desired gene on its chromosome or in its genome. Many host cells that can be employed in the practice of the present invention (e.g., CHO cells) serve as hosts are well known in the art. See, e.g., Sambrook et al., Molecular Cloning: A Laboratory Manual, Cold Spring Harbor Press (3^{rd} ed., 2001); and Brent et al., Current Protocols in Molecular Biology, John Wiley & Sons, Inc. (ringbou ed., 2003). In some preferred embodiments, the host cell is a mammalian cell.

The term “operably linked” or “operably associated” refers to functional linkage between genetic elements that are joined in a manner that enables them to carry out their normal functions. For example, a gene is operably linked to a promoter when its transcription is under the control of the promoter and the transcript produced is correctly translated into the protein normally encoded by the gene. Similarly, a HH ribozyme variant-coding sequence is operably linked to a target gene if its insertion into the 5′-UTR or 3′-UTR of the gene, as described herein, allows control of the target gene expression by the ribozyme in the presence or absence of an inhibitor of the ribozyme.

A “substantially identical” nucleic acid or amino acid sequence refers to a polynucleotide or amino acid sequence which comprises a sequence that has at least 75%, 80% or 90% sequence identity to a reference sequence as measured by one of the well known programs described herein (e.g., BLAST) using standard parameters. The sequence identity is preferably at least 95%, more preferably at least 98%, and most preferably at least 99%. In some embodiments, the subject sequence is of about the same length as compared to the reference sequence, i.e., consisting of about the same number of contiguous amino acid residues (for polypeptide sequences) or nucleotide residues (for polynucleotide sequences).

Sequence identity can be readily determined with various methods known in the art. For example, the BLASTN program (for nucleotide sequences) uses as defaults a wordlength (W) of 11, an expectation (E) of 10, M=5, N=-4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)). Percentage of sequence identity is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

A cell has been “transformed” or “transfected” by exogenous or heterologous polynucleotide when such polynucleotide has been introduced inside the cell. The transforming polynucleotide may or may not be integrated (covalently linked) into the genome of the cell. In prokaryotes, yeast, and mammalian cells for example, the transforming polynucleotide may be maintained on an episomal element such as a plasmid. With respect to eukaryotic cells, a stably transformed cell is one in which the transforming polynucleotide has become integrated into a chromosome so that it is inherited by daughter cells through chromosome replication. This stability is demonstrated by the ability of the eukaryotic cell to establish cell lines or clones comprised of a population of daughter cells containing the transforming polynucleotide. A “clone” is a population of cells derived from a single cell or common ancestor by mitosis. A “cell line” is a clone of a primary cell that is capable of stable growth in vitro for many generations.

The term “vector” or “construct” refers to polynucleotide sequence elements arranged in a definite pattern of organization such that the expression of genes/gene products that are operably linked to these elements can be predictably controlled. Typically, they are transmissible polynucleotide sequences (e.g., plasmid or virus) into which a segment of foreign polynucleotide sequence can be spliced in order to introduce the foreign DNA into host cells to promote its replication and/or transcription.

A “vector” or “construct” is a nucleic acid with or without a carrier that can be introduced into a cell. Vectors capable of directing the expression of heterologous polynucleotide or target gene sequences encoding for one or more polypeptides are referred to as “expression vectors” or “expression constructs”. The cloned target gene sequence is usually placed under the control of (i.e., operably linked to) certain regulatory sequences such as promoters, enhancers and ribozyme-coding sequences.

As used herein, “AAV” is adeno-associated virus, and may be used to refer to the naturally occurring wild-type virus itself or derivatives thereof. The term covers all subtypes, serotypes and pseudotypes, and both naturally occurring and recombinant forms, except where required otherwise. Pseudotyped AAV refers to an AAV that contains capsid proteins from one serotype and a viral genome including 5′-3′ ITRs of a second serotype. The abbreviation “rAAV” refers to recombinant adeno-associated viral particle or a recombinant AAV vector (or “rAAV vector”). An “AAV virus” or “AAV viral particle” refers to a viral particle composed of at least one AAV capsid protein (preferably by all of the capsid proteins of a wild-type AAV) and an encapsidated polynucleotide. If the particle comprises a heterologous polynucleotide (i.e., a polynucleotide other than a wild-type AAV genome such as a transgene to be delivered to a mammalian cell), it is typically referred to as “rAAV”.

A retrovirus (e.g., a lentivirus) based vector or retroviral vector means that genome of the vector comprises components from a retrovirus or lentivirus as a backbone. The viral particle generated from the vector as a whole contains essential vector components compatible with the RNA genome, including reverse transcription and integration systems. Usually these will include the gag and pol proteins derived from the virus. If the vector is derived from a lentivirus, the viral particles are capable of infecting and transducing non-dividing cells. Recombinant retroviral particles are able to deliver a selected exogenous gene or polynucleotide sequence such as therapeutically active genes, to the genome of a target cell.

### III. Modified and Optimized Hammerhead Ribozymes with Improved Dynamic Range

The present invention provides RNA based on-switches that contain an optimized HH ribozyme variant with increased dynamic range. Dynamic range of a ribozyme or RNA based on-switch refers to the extent of expression regulation afforded by the ribozyme on a target sequence that is placed under the control of (i.e., operably linked to) the ribozyme. This can be calculated as, e.g., fold inhibition in the target gene expression when placed under control of the ribozyme relative to its expression without the ribozyme or when placed under control of an inactive ribozyme.

HH ribozymes are RNA motifs that are capable of sustaining either in trans or in cis cleavage of a phosphodiester bond. See Scott et al., Prog. Mol. Biol. Transl. Sci. 120: 1-23, 2013. A cis-acting HH ribozyme is a catalytic RNA that undergoes self-cleavage of its own backbone to produce two RNA products. Cis-acting HH ribozymes contain three base-paired stems and a highly conserved core of residues required for cleavage. The cleavage reaction proceeds by an attack of a 2′ hydroxyl oxygen of a catalytic site cytosine on the phosphorus atom attached to the 3′ carbon of the same residue. This breaks the sugar phosphate backbone and produces a 2′,3′ cyclic phosphate. The minimal HH sequence that is required for the self-cleavage reaction includes approximately 13 conserved or invariant “core” nucleotides, most of which are not involved in forming canonical Watson-Crick base-pairs. The core region is flanked by stems I, II and III, which are in general comprised of canonical Watson-Crick base-pairs but are otherwise not constrained with respect to sequence. Some HH ribozymes cleave the target sequence in trans. Cleavage specificity of a trans-acting HH ribozyme is controlled by the hybridizing arms of the ribozyme, which anneal with the substrate in a complementary fashion and direct cleavage of the scissile phosphodiester bond. This activity is specifically directed to occur after the third nucleotide of the cleavage triplet.

Preferably, engineered cis-acting HH ribozymes are used in constructing the RNA on switches and related expression vectors of the invention. Typically, the engineered ribozymes of the invention are type III HH ribozyme variants that are modified from a known cis-acting type I HH ribozyme. Type III HH ribozymes are modified cis-acting ribozymes that have the 5′ and 3′ ends in stem III. This is in contrast to the type I HH ribozymes, which have its 5′ and 3′ ends in stem I. Specific examples of type I HH ribozyme for engineering type III variants are exemplified herein, e.g., ribozymes N107 and N117 which are well-known in the art (see, e.g., Yen et al., Nature 431, 471-476, 2004). In addition to the well-known modified Schistosoma mansoni HH ribozymes N107 and N117, many other modified or natural HH ribozymes (including natural Schistosoma mansoni HH ribozymes) are also known in the art. See, e.g., Ferbeyre et al., Mol. Cell. Biol. 18:3880-3888, 1998; Chartrand et al., Nucleic Acids Res. 23:4092-4096, 1995; and Canny et al., Biochemistry 46:3826-3834, 2007.

In some embodiments, the type III HH ribozyme variants of the invention can be engineered from any modified or natural Schistosoma mansoni hammerhead ribozymes. In some of these embodiments, the type III HH ribozyme variants of the invention are generated from modified (or engineered) type I Schistosoma mansoni HH ribozymes N107 or N117. As demonstrated herein with ribozyme variant T3H1 (SEQ ID NO:8), such a type III conversion can lead to an increase of dynamic range of regulation from ˜18 fold observed with the type I enzyme N107 to ˜134 fold. Conversion of a type I HH ribozyme into a type III variant can be readily accomplished as described herein or via standard techniques of molecule biology. In addition to the type III conversion, the HH ribozyme variants of the invention further contain one or more modifications in the sequences that results in further improvement of dynamic range of their enzymatic activities. These additional modifications include, e.g., optimization of stem III of the resulting type III ribozymes, modification of stem I to stabilize the tertiary interactions essential for ribozyme function, and alteration of loop I to facilitate hairpin formation. As exemplified herein, successive modifications introduced into type I HH ribozyme N107 led to an increase of dynamic range from its original ˜18 fold to ˜1,200 fold.

In some embodiments, the type III HH ribozymes of the invention contain an optimized stem III relative to the type I enzyme from which they are derived. Optimization of stem III sequence is intended to balance cleavage and disassembly rates. A more stable stem III facilitates cleavage but slows disassembly, whereas a less stable stem III works in the opposite direction. Thus, an optimal stem III sequence that balances these processes affords the maximum inhibition of gene expression. As exemplified herein, the optimization involves designing stem III of varying lengths and/or with varied base locations.

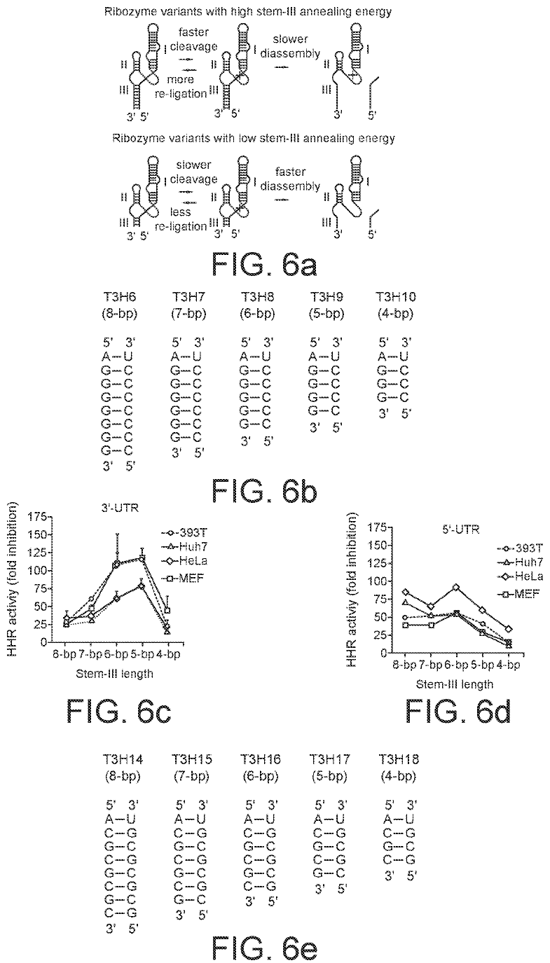

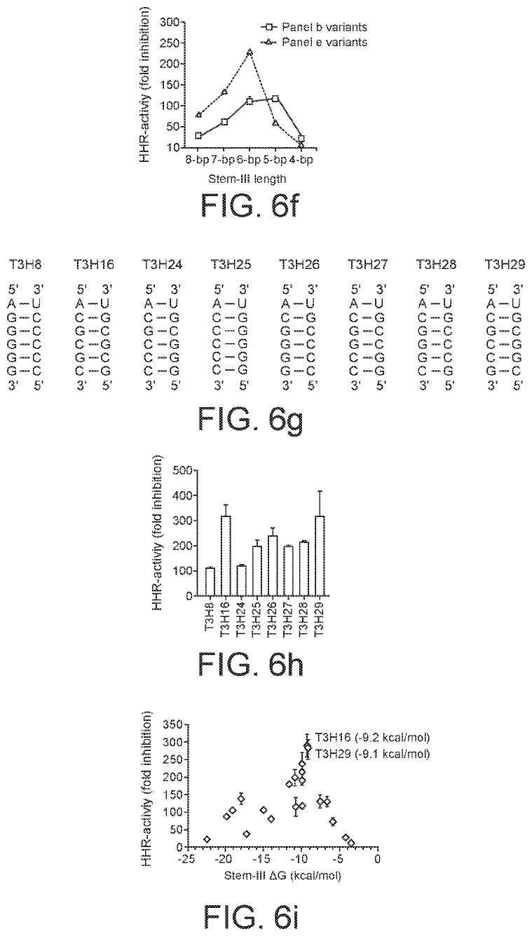

As exemplified herein, the optimized stem III contains from about 4 to about 10 base pairs. In various embodiments, the optimized stem III contains 4, 5, 6, 7, or 8 base pairs. In some preferred embodiments, the optimized stem III contains 5 or 6 base pairs. In addition to an appropriate length, the optimized stem III should also contain appropriate base pairings that result in a calculated annealing energy of about −9 kcal/mol, as exemplified herein with ribozyme variants T3H16 and T3H29. Annealing energy calculation can be performed as described in the art. See, e.g., Bellaousov et al., RNAstructure: Web servers for RNA secondary structure prediction and analysis. Nucleic Acids Res 41, W471-474, 2013. Some specific examples of optimized stem III sequences are described herein (FIG. 6). These include HH ribozyme variants T3H16 (SEQ ID NO:9) and T3H29 (SEQ ID NO:10), which respectively have the two sequences on the opposite sides of the optimized stem III (5′-3′/3′-5′) acgcgc/ugcgcg (T3H16) or acgccg/ugcggc (T3H29).

Sequence of HH ribozyme variant T3H16: gcgcg tcctg gattc cactg ctatc cattc gtgag gtgca ggtac atcca gctga cgagt cccaa atagg acgaa acgcg c (SEQ ID NO:9).

Sequence of HH ribozyme variant T3H29: cggcg tcctg gattc cactg ctatc cattc gtgag gtgca ggtac atcca gctga cgagt cccaa atagg acgaa acgccg (SEQ ID NO:10)

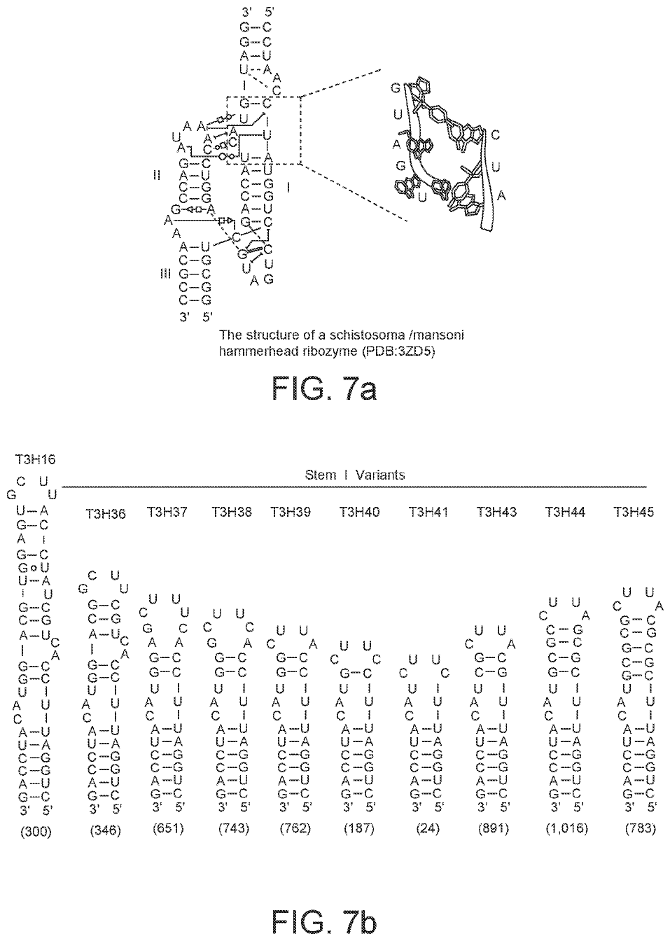

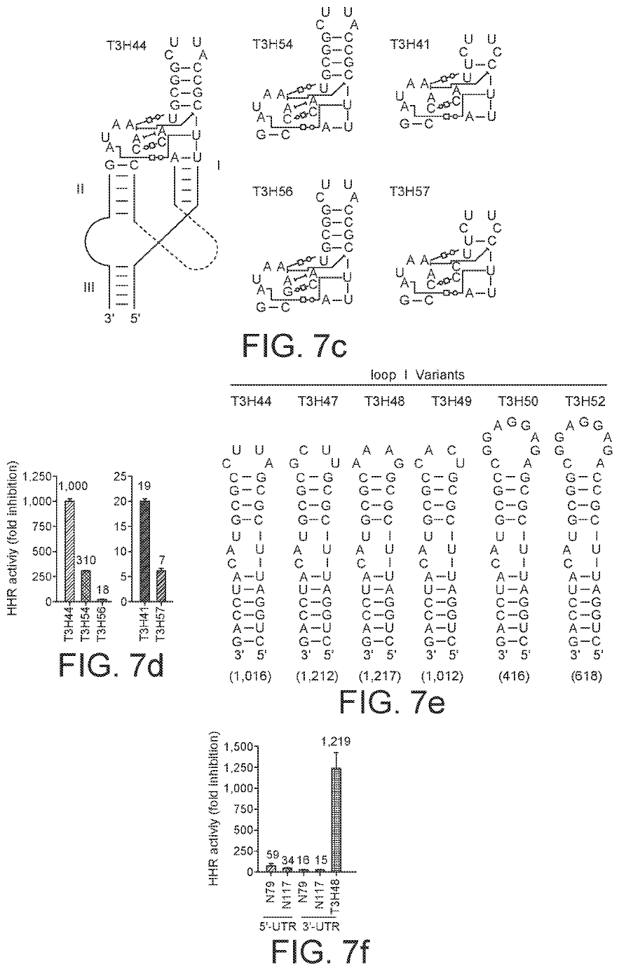

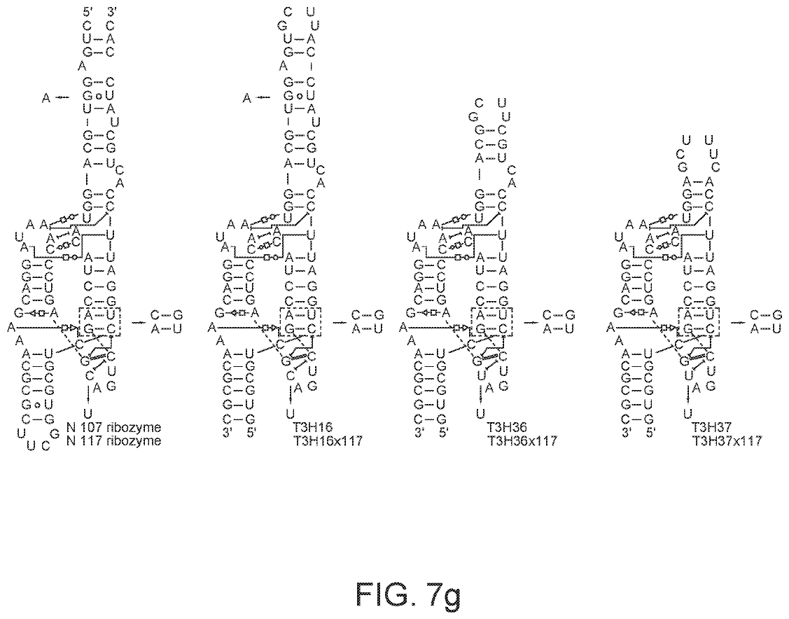

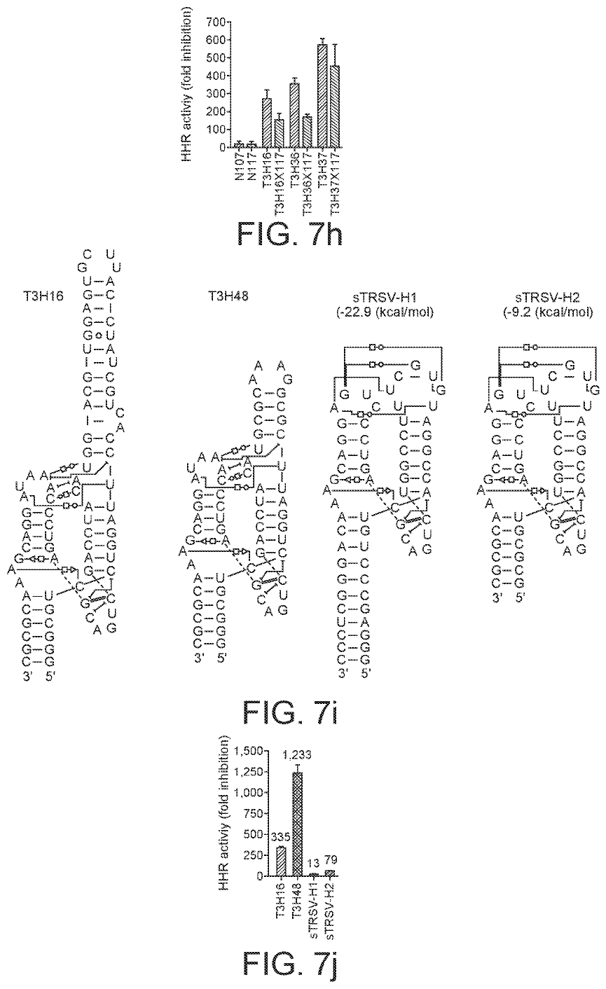

Additionally, the type III HH ribozyme variants of the invention can contain a modified stem I relative to that of the type I HH ribozyme from which they are derived. Stem I has a “UAC” bulge, which forms tertiary interactions with loop II and accelerate HH ribozyme self cleavage by 100-1000 fold, as described in the art. See, e.g., Khvorova et al., Nat Struct Biol 10, 708-712, 2003; De la Pena et al., EMBO J. 22, 5561-5570, 2003; and Martick et al., Cell 126, 309-320, 2006. The modification in stem I is intended to promote the “UAC” bulge formation, thus to facilitate the tertiary interactions between stem I and loop II as described herein. Such modifications can include, e.g., introducing perfectly matched residues to form a new stem immediately 5′ to the “UAC” bulge, and introduction of a stable tetraloop to stabilize the new stem, as exemplified herein in ribozyme variant T3H44 and T3H48. Specific examples of HH ribozyme variants that contain such a modified stem I, in addition to an optimized stem III described above, include variants T3H36 (SEQ ID NO:11), T3H37 (SEQ ID NO:12), T3H38 (SEQ ID NO:13), T3H39 (SEQ ID NO:14), T3H43 (SEQ ID NO:15), T3H44 (SEQ ID NO:16) and T3H45 (SEQ ID NO:17), as well as T3H47 (SEQ ID NO:18), T3H48 (SEQ ID NO:19) and T3H49 (SEQ ID NO:20) described below. Variants of these specific enzymes with substantially identical sequences or conservatively modified residues are also encompassed by the invention. Structures of the stem I/loop I regions of some of these ribozymes are shown in FIG. 7b. In various embodiments, the two sequences on the opposite sides of the modified stem I, immediately 5′ to the “UAC” bulge, can be (5′-3′/3′-5′) cc/gg (as exemplified in enzymes T3H36-T3H39), cgc/gcg (as exemplified in enzyme T3H43), cgcg/gcgc (as exemplified in enzymes T3H44 and T3H47-T3H49), or cgcgc/gcgcg (as exemplified in enzyme T3H45).

Sequences of some of the exemplified HH ribozyme variants:

T3H36:
(SEQ ID NO: 11)
gcgcg tcctg gattc cactg cttcg gcagg tacat ccagc
tgacg agtcc caaat aggac gaaac gcgc.
T3H37:
(SEQ ID NO: 12)
gcgcg tcctg gattc cactt tcgag gtaca tccag ctgac
gagtc ccaaa tagga cgaaa cgcgc.
T3H38:
(SEQ ID NO: 13)
gcgcg tcctg gattc cactt cgggt acatc cagct gacga
gtccc aaata ggacg aaacg cgc.
T3H39:
(SEQ ID NO: 14)
gcgcg tcctg gattc cattc ggtac atcca gctga cgagt
cccaa atagg acgaa acgcg c.
T3H43:
(SEQ ID NO: 15)
gcgcg tcctg gattc gcatt cgcgt acatc cagct gacga
gtccc aaata ggacg aaacg cgc.
T3H44:
(SEQ ID NO: 16)
gcgcg tcctg gattc gcgat tccgc gtaca tccag ctgac
gagtc ccaaa tagga cgaaa cgcgc.
T3H45:
(SEQ ID NO: 17)
gcgcg tcctg gattc gcgca ttcgc gcgta catcc agctg
acgag tccca aatag gacga aacgc gc.

Additionally, the type III HH ribozyme variants of the invention can further contain a stable tetraloop to facilitate hairpin formation. The alteration in loop I is intended again to promote the “UAC” bulge formation, thus to facilitate the tertiary interactions between stem I and loop II. As demonstrated herein, by changing loop I residues to make more stable tetraloops, further improvement in ribozyme activity can be achieved. As exemplified with HH ribozyme variant T3H48, loop I sequence is changed from auuc to gaaa in the type III variant T3H44. Examples of stable tetraloop sequences have been described in the art. See, e.g., Bottaro S and Lindorff-Larsen K, Biophys J 113, 257-267, 2017. In various embodiments, the altered loop I sequence in the HH ribozyme variants of the invention can be auuc (exemplified in enzyme T3H44), uucg (exemplified in enzyme T3H47), gaaa (exemplified in enzyme T4H48), ucac (exemplified in enzyme T3H49), or agaggaggc (exemplified in enzymes T3H50 and TH3H52), as illustrated in FIG. 7e.

Sequence of HH ribozyme variant T3H47:
(SEQ ID NO: 18)
gcgcg tcctg gattc gcgtt cgcgc gtaca
tccag ctgac gagtc ccaaa tagga cgaaa cgcgc.
Sequence of HH ribozyme variant T3H48:
(SEQ ID NO: 19)
gcgcg tcctg gattc gcgga aacgc gtaca
tccag ctgac gagtc ccaaa tagga cgaa acgcg c.
Sequence of HH ribozyme variant T3H49:
(SEQ ID NO: 20)
gcgcg tcctg gattc gcgtc accgc gtaca
tccag ctgac gagtc ccaaa tagga cgaaa cgcgc.
Sequence of HH ribozyme variant T3H50:
(SEQ ID NO: 21)
gcgcg tcctg gattc gcgag aggag gccgc
gtaca tccag ctgac gagtc ccaaa tagga cgaaa cgcgc.
Sequence of HH ribozyme variant T3H52:
(SEQ ID NO: 22)
gcgcg tcctg gattc ggcca gagga ggcgg ccgta
catcc agctg acgag tccca aatag gacga aacgc gc .

Production of ribozyme variants of the invention can be carried out in accordance with the methods exemplified herein or molecule biology methods routinely practiced in the art. For example, they can be produced by recombinant nucleic acid techniques. Endogenous RNA polymerase of the treated cell may mediate transcription in vivo, or cloned RNA polymerase can be used for transcription in vitro. Ribozyme variants of the invention may include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of nitrogen or sulfur heteroatom. Modifications in RNA structure may be tailored to allow specific genetic inhibition while avoiding a general response to dsRNA. Likewise, bases may be modified to block the activity of adenosine deaminase. Various methods known in the art for chemically modifying RNA molecules can also be adapted for modifying the HH ribozyme variants of the invention. See, e.g., Heidenreich et al., Nucleic Acids Res, 25:776-780, 1997; Wilson et al., J Mol. Recog. 7:89-98, 1994; Chen et al., Nucleic Acids Res 23:2661-2668, 1995; and Hirschbein et al., Antisense Nucleic Acid Drug Dev 7:55-61, 1997.

### IV. Expression Vector Harboring Ribozyme Variants of the Invention

As exemplified herein, the HH ribozyme variants with improved dynamic range of expression regulation as described herein can be readily employed to control expression of a target gene expression in a target cell, in combination with the use of a morpholino oligonucleotide inducer. The invention accordingly provides expression constructs that harbor a target gene that is operably linked to a HH ribozyme variant of the present invention. Such an inserted ribozyme can inhibit translation by self-cleavage of the mRNA transcript expressed from the vector. Typically, the sequence encoding a HH ribozyme variant described herein can be inserted into the target gene at a non-coding region that is important for translation. The insertion site can be at a position that lies in any of the untranslated regions that are known to be involved in controlling mRNA translation, degradation, and/or localization. These include, e.g., stem-loop structures, upstream initiation codons and open reading frames, internal ribosome entry sites and various cis-acting elements that are bound by RNA-binding proteins. In the absence of the morpholino oligonucleotide inducer, the ribozyme undergoes self-cleavage, resulting degradation of the mRNA and inhibition of translation.

In some embodiments, the ribozyme coding sequence (e.g., encoding ribozyme T3H38, T3H44 or T3H52 exemplified herein) can be inserted at 3′-untranslated region (3′-UTR). Regulatory regions within the 3′-untranslated region can influence polyadenylation, translation efficiency, localization, and stability of the mRNA. The 3′-UTR contains both binding sites for regulatory proteins as well as microRNAs (miRNAs). The 3′-UTR also has silencer regions which bind to repressor proteins and will inhibit the expression of the mRNA. Many 3′-UTRs also contain AU-rich elements (AREs). Proteins bind AREs to affect the stability or decay rate of transcripts in a localized manner or affect translation initiation. Furthermore, the 3′-UTR contains the sequence aauaaa that directs addition of several hundred adenine residues called the poly(A) tail to the end of the mRNA transcript. Poly(A) binding protein (PABP) binds to this tail, contributing to regulation of mRNA translation, stability, and export. For example, poly (A) tail bound PABP interacts with proteins associated with the 5′ end of the transcript, causing a circularization of the mRNA that promotes translation. Cleavage by the inserted ribozyme at any of these regions in the 3′-UTR can lead to disruption or suppression of mRNA translation.

In some other embodiments, a sequence encoding the ribozyme can be inserted at a site that lies in the 5′-untranslated region (5′-UTR) of the corresponding mRNA transcript. The 5′-UTR, also known as a leader sequence or leader RNA, is the region of an mRNA that is directly upstream from the initiation codon. This region is important for the regulation of translation of a transcript by differing mechanisms in viruses, prokaryotes and eukaryotes. While called untranslated, the 5′-UTR or a portion of it is sometimes translated into a protein product. This product can then regulate the translation of the main coding sequence of the mRNA. In many other organisms, however, the 5′-UTR is completely untranslated, instead forming complex secondary structure to regulate translation. Regulatory elements within 5′-UTRs have also been linked to mRNA export. By inserting the ribozyme at an appropriate site in the 5′-UTR corresponding region of the target gene, expression of the target gene can be efficiently controlled with a morpholino inducer that specifically inhibits the ribozyme in the expression construct.

The expression constructs of the invention are typically circular vectors and, in addition to the ribozyme sequence and optionally an operably linked target gene, can also contain selectable markers, an origin of replication, and other elements. For example, the vector can contain a selection marker. The selection marker allows one to select for cells into which the vector has been introduced and/or stably integrated. In some embodiments, the selection marker can be a polynucleotide encoding a protein or enzyme that confers to the cells visually identifiable characteristics. For example, the vector can harbor a selection marker encoding Renilla luciferase reporter enzyme. Other examples include jellyfish green fluorescent protein (GFP) and bacterial β-galactosidase. In some other embodiments, the selection marker for identifying host cells into which the vector was introduced and/or stably integrated can be an antibiotic resistance gene. Examples of such markers include antibiotic resistance genes for neomycin, chloramphenicol, blasticidin, hygromycin, and zeocin. he expression vectors of the invention can also bear other DNA sequences that may be necessary for proper RNA transcription and processing, as well as proper ribosome assembly and function. For example, some vectors of the invention additionally harbor sequences corresponding to the 5′-ETS and ITS elements of the precursor RNA sequence.

To control a specific target gene expression, the target gene is typically operably fused with a sequence encoding a HH ribozyme variant described herein in the expression construct. Depending on the specific cells into which the target gene is to be introduced, a suitable expression construct is first to be generated with the target gene and the ribozyme coding sequence. For controlling gene expression in mammalian cells, the expression constructs can be recombinantly produced with many vectors well known in the art. These include viral vectors such as recombinant adenovirus, retrovirus, lentivirus, herpesvirus, poxvirus, papilloma virus, or adeno-associated virus. The vectors can be present in liposomes, e.g., neutral or cationic liposomes, such as DOSPA/DOPE, DOGS/DOPE or DMRIE/DOPE liposomes, and/or associated with other molecules such as DNA-anti-DNA antibody-cationic lipid (DOTMA/DOPE) complexes. Exemplary viral vectors suitable for the invention are described herein. For in vivo application, the expression vectors may be administered to a subject via any route including, but not limited to, intramuscular, buccal, rectal, intravenous or intracoronary routes.

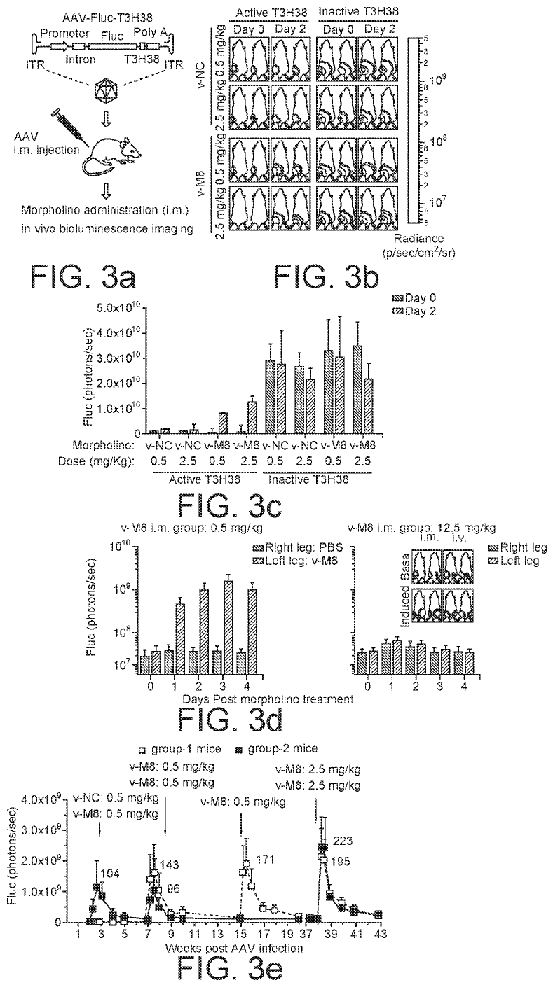

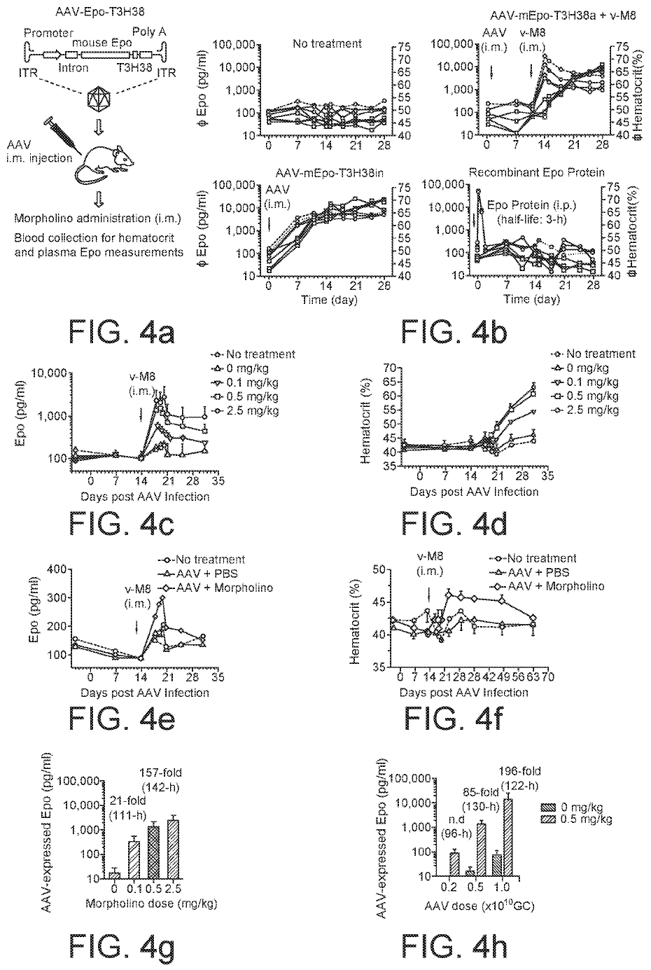

In some preferred embodiments the expression constructs are based on adeno-associated viral (AAV) vectors or adenoviral vectors. Adeno-associated virus (AAV) is a small, nonenveloped virus that was adapted for use as a gene transfer vehicle. Adeno-associated virus vectors refer to recombinant adeno-associated viruses that are derived from nonpathogenic parvoviruses. They evoke essentially no cellular immune response, and produce transgene expression lasting months in most systems. Like adenovirus, adeno-associated virus vectors also have the capability to infect replicating and nonreplicating cells and are believed to be nonpathogenic to humans. Delivery of heterologous polynucleotide sequences via recombinant AAV can provide for safe, unobtrusive and sustained expression (>2 year) of high levels of protein therapeutics. As exemplification, the invention provides AAV vectors which are used for controlling expression of a reporter gene (e.g., firefly luciferase) or a therapeutic protein (e.g., Epo). In these vectors, the target gene (Fluc or Epo) is operably fused to a HH ribozyme variant described herein (e.g., T3H38). As demonstrated herein (e.g., FIGS. 3 and 4), these expression constructs allow control of transgene expression in vivo in a manner that is dose-dependent on the presence of the morpholino inducer.

In some embodiments, the expression constructs are based on retroviral vectors. In order to construct a retroviral vector for gene transfer, the target gene and the ribozyme coding sequence (which is fused with the target gene at, e.g., the 3′-UTR or 5′-UTR region) are inserted into the viral genome in the place of certain viral sequences to produce a viral construct that is replication-defective. In order to produce virions, a producer host cell or packaging cell line is employed. The host cell usually expresses the gag, pol, and env genes but without the LTR and packaging components. When the recombinant viral vector containing the gene of interest together with the retroviral LTR and packaging sequences is introduced into a host cell (e.g., by calcium phosphate precipitation), the packaging sequences allow the RNA transcript of the recombinant vector to be packaged into viral particles, which are then secreted into the culture media. The media containing the recombinant retroviruses can then be collected, optionally concentrated, and used for transducing host cells (e.g., stem cells) in gene transfer applications. Suitable host or producer cells for producing recombinant retroviruses or retroviral vectors according to the invention are well known in the art (e.g., 293T cells exemplified herein). Many retroviruses have already been split into replication defective genomes and packaging components. For other retroviruses, vectors and corresponding packaging cell lines can be generated with methods routinely practiced in the art. The producer cell typically encodes the viral components not encoded by the vector genome such as the gag, pol and env proteins. The gag, pol and env genes may be introduced into the producer cell and stably integrated into the cell genome to create a packaging cell line. The retroviral vector genome is then introduced-into the packaging cell line by transfection or transduction to create a stable cell line that has all of the DNA sequences required to produce a retroviral vector particle. Another approach is to introduce the different DNA sequences that are required to produce a retroviral vector particle, e.g. the env coding sequence, the gag-pol coding sequence and the defective retroviral genome into the cell simultaneously by transient triple transfection. Alternatively, both the structural components and the vector genome can all be encoded by DNA stably integrated into a host cell genome.

Expression vectors harboring a target gene sequence and an operably linked ribozyme-coding sequence can be readily constructed in accordance with methodologies known in the art of molecular biology in view of the exemplifications provided herein specification. See, e.g., Sambrook et al., Molecular Cloning: A Laboratory Manual, Cold Spring Harbor Press (3^{rd} ed., 2001); Brent et al., Current Protocols in Molecular Biology, John Wiley & Sons, Inc. (ringbou ed., 2003); and Freshney, Culture of Animal Cells: A Manual of Basic Technique, Wiley-Liss, Inc. (4^{th} ed., 2000). Typically, the expression vectors are assembled by inserting into a suitable vector backbone the polynucleotide encoding the target gene and ribozyme sequence fusion, sequences encoding selection markers, and other optional elements. Many virus based expression vector systems well known in the art can be used in the invention. Widely used retroviral vectors include those based upon murine leukemia virus (MuLV), gibbon ape leukemia virus (GaLV), simian immunodeficiency virus (SIV), human immunodeficiency virus (HIV), and combinations thereof (see, e.g., Buchscher et al., J. Virol. 66:2731-2739, 1992; Johann et al., J. Virol. 66:1635-1640, 1992; Sommerfelt et al., Virol. 176:58-59, 1990; Wilson et al., J. Virol. 63:2374-2378, 1989; Miller et al., J. Virol. 65:2220-2224, 1991; and PCT/US94/05700). Adeno-associated viral vectors have also been used in many reported studies for gene therapy in research and clinical environment. See, e.g., Kaplitt et al., Lancet 369: 2097-105, 2007; Daya et al., Clin Microbiol Rev. 21(4): 583-593, 2008; Strobel et al., Am. J. Resp. Cell Mol. Biol. 53: 291-302, 2015; and Kotterman et al., Nat. Rev. Genet. 15:445-451, 2014. Many viral vectors and related reagents (e.g., packaging cell lines) suitable for the invention can be obtained commercially. For example, AAV based expression vectors for practicing the invention can be based on the pAAV-MCS construct that is available from Agilent Technologies (Santa Clara, Calif.). Similarly, a number of retroviral vectors and compatible packing cell lines are available from Clontech (Mountain View, Calif.). Examples of lentiviral based vectors include, e.g., pLVX-Puro, pLVX-IRES-Neo, pLVX-IRES-Hyg, and pLVX-IRES-Puro. Corresponding packaging cell lines are also available, e.g., Lenti-X 293T cell line. In addition to lentiviral based vectors and packaging system, other retroviral based vectors and packaging systems are also commercially available. These include MMLV based vectors pQCXIP, pQCXIN, pQCXIQ and pQCXIH, and compatible producer cell lines such as HEK 293 based packaging cell lines GP2-293, EcoPack 2-293 and AmphoPack 293, as well as NIH/3T3-based packaging cell line RetroPack PT67. Any of these and other retroviral vectors and producer cell lines may be employed in the practice of the present invention.

### V. Activate Target Gene Expression from Vectors Harboring a Ribozyme Variant

The various HH ribozyme variants described herein provide functional RNA on-switches to regulate gene expression in a target cell. Typically, the target cell is engineered to harbor an expression construct that includes a coding sequence for the target gene that is operably linked to a sequence encoding a HH ribozyme variant of the invention. Upon transcription, translation of the encoded target polypeptide molecule is inhibited as a result of enzymatic cleavage of the RNA transcript by the cis-acting ribozyme. To activate target gene expression, an inhibitor molecule or expression inducer that specifically target the ribozyme is employed.

Preferably, the inhibitor molecule used for activating target gene expression is an RNase H independent antisense oligonucleotide, such as a morpholino based oligomeric compound, a 2′-O-methoxyethyl phosphorothioate-modified antisense oligonucleotide, or a locked nucleic acid (LNA)/DNA mixmer that contains an oligomer (oligonucleotide) that specifically targets the ribozyme sequence in the expression construct. Morpholino-based oligomeric compounds are non-ionic mimetics of oligonucleotides. They are different from natural nucleic acids, with methylenemorpholine rings replacing the ribose or deoxyribose sugar moieties and non-ionic phosphorodiamidate linkages replacing the anionic phosphates of DNA and RNA. Each morpholine ring suitably positions one of the standard DNA bases (A, C, G, T) for pairing, so that a 25-base Morpholino oligo strongly and specifically binds to its complementary 25-base target site in a strand of RNA via Watson-Crick pairing. Because the uncharged backbone of the Morpholino oligo is not recognized by enzymes, it is completely stable to nucleases. Morpholino oligonucleotides with various different structures and linking groups are described in the art. See, e.g., Dwaine et al., Biochemistry, 2002, 41, 4503-4510; Heasman et al., J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. Nos. 5,698,685, 5,217,866, 5,142,047, 5,034,506, 5,521,063, 5,506,337, 5,034,506, 5,166,315, and 5,185,444.

Morpholino oligos are advanced tools for blocking sites on RNA to obstruct cellular processes. A Morpholino oligo specifically binds to its selected target site to block access of cell components to that target site. This property can be exploited to block translation, block splicing, block miRNAs or their targets, and block ribozyme activity. Like all gene knockdown reagents, Morpholinos must be actively delivered into most cells. Morpholinos can be delivered into cultured cells by a variety of methods, including scrape-loading of adherent cells, electroporation, and even microinjection. As exemplified herein, Endo-Porter delivery reagents (Gene Tools, LLC; Philomath, Oreg.) can be used to achieve excellent delivery in cultured cells in terms of the amount of Morpholino delivered per cell, even distribution throughout a population of cells, reproducibility of delivery and non-toxicity for most cell types at the recommended concentration.

Provided that they are modified to contain an anti-sense oligonucleotide that is complementary to a HH ribozyme variant described herein (e.g., T3H38 or T3H44), many Morpholino oligos known in the art and readily available from commercial vendors can be employed in the practice of the invention. These include unmodified morpholino oligos as well as modified morpholino compounds such as photo-morpholinos and vivo-morpholinos. In various embodiments, the invention can employ phosphorodiamidate morpholino oligonucleotides for suppressing the ribozyme on-switch described herein and thereby inducing gene expression. In some preferred embodiments, the invention utilize vivo-morpholinos to induce gene expression from an expression vector harboring the modified ribozyme on-switch described herein. Vivo-morpholinos are the knockdown, exon-skipping or miRNA blocking reagent of choice for in vivo experiments. See, e.g., Morcos et al., Biotechniques 45, 613-614, 2008; and Stein et al., Mol Ther 25, 1069-1075, 2017.

A vivo-morpholino is comprised of a Morpholino oligo with a unique covalently linked delivery moiety, which is comprised of an octa-guanidine dendrimer as exemplified herein. It uses the active component of arginine rich delivery peptides (the guanidinium group) with improved stability and reduced cost. The Vivo-Morpholino is assembled by coupling the vivo-delivery group to a Morpholino while the oligo is still bound to its synthesis resin, allowing excellent purification by washing the solid-phase resin. Vivo-Morpholinos must be chosen prior to synthesis and cannot be added later because the vivo-delivery group is added to a Morpholino prior to cleavage from its synthesis resin.

As described herein, the morpholino based compounds (morpholino oligonucleotides or vivo-morpholinos) used in the invention should typically contain sequences that are complementary to the ribozyme switches that are present in the expression vectors. In some embodiments, the morpholino compounds contain complementary sequences that target the 5′-region of the ribozyme. Specific examples of morpholino compounds that can be used in combination with the ribozyme switches described herein include vivo-morpholinos M2-M10. These vivo-morpholinos, in addition to the coupled octa-guanidine dendrimer, contain an oligonucleotide that has a sequence shown in SEQ ID NOs:38-46, respectively. As detailed herein, these vivo-morpholinos can be used to induce gene expression from vectors that contain any of the variant HH ribozymes exemplified herein, e.g., T3H16, T3H38, T3H48 and T3H52. The morpholino compounds that specifically target and inactivate a ribozyme switch can be readily synthesized de novo via standard protocols of organic chemistry and molecule biology. See, e.g., Mardirossian et al., J. Nucl. Med. 38:907-13, 1997; Summerton et al., Biochim. Biophys. Acta 1489, 141-158, 1999; Morcos et al., Biotechniques 45, 613-614, 2008; WO2014052276A1; and U.S. Pat. No. 6,899,864. Alternatively, they can be custom synthesized by commercial suppliers, e.g., Gene Tools, LLC (Philomath, Oreg.) and Sarepta Therapeutics (Cambridge, Mass.).

### VI. Applications of RNA Switches of the Invention in Gene Expression Control

The HH ribozyme variants with improved dynamic range described herein and expression vectors containing the ribozymes, in combination with the use of a morpholino oligonucleotide (or morpholino based compound) inducer, can be employed to control expression of proteins and protein biologics in various scientific and medical contexts, e.g., to afford simple and immediate regulation of gene expression in cell biology studies. In some embodiments, they can be used to transform gene therapy by allowing controlled dosing of a biologic with a well tolerated drug. In these applications, the expression systems of the invention have several important advantages. The size of the regulatory element is about 63 bp, small enough, for example, to control expression of a CRISPR effector protein and guide RNA in the same AAV vector, minimizing off-target activity and immunogenicity in therapeutic settings. Moreover, roughly half of unregulated transgene expression can be recovered. It is therefore useful in contexts, such as therapeutic antibodies, where high expression is paramount. The system is promoter-independent, allowing tissue-specific induction. Local administration and induction allow for independent regulation of two or more therapeutics in the same individual. As demonstrated herein, morpholino induction can last for weeks, significantly improving the half-lives of short-lived proteins or peptides. Finally, some of these properties can be useful for regulating survival, payload expression, or local activation of cell-based gene therapies, or for regulating the replication of live vaccines or oncolytic viruses.

Controlling target gene expression via the RNA based switches of the invention can be used in various clinical or industrial applications. The recombinant expression vectors expressing a target gene or heterologous polynucleotide sequence can be transduced into host cells in various gene therapy and industrial bioengineering settings. For example, the target gene or heterologous polynucleotide sequence harbored by the recombinant expression vectors can encode a therapeutic agent. These constructs can be transferred, e.g., to express a biologically important agent that is otherwise present in normal healthy subjects (e.g., Epo as exemplified herein), to express a protein drug to treat various diseases (e.g., cancer), to express immunomodulatory genes to fight viral infections, or to replace a gene's function as a result of a genetic defect. In various embodiments, the target gene can encode one of the many therapeutic proteins known in the art. These include erythropoietin (Epo), insulin, factor VIII, factor IX, β-globin, low-density lipoprotein receptor, adenosine deaminase, purine nucleoside phosphorylase, sphingomyelinase, glucocerebrosidase, cystic fibrosis transmembrane conductance regulator, α-antitrypsin, CD-18, ornithine transcarbamylase, argininosuccinate synthetase, phenylalanine hydroxylase, branched-chain α-ketoacid dehydrogenase, fumarylacetoacetate hydrolase, glucose 6-phosphatase, α-L-fucosidase, β-glucuronidase, α-L-iduronidase, galactose 1-phosphate uridyltransferase, interleukins, cytokines, small peptides, and the like. Other therapeutic proteins that can be expressed from an intergrated target polynucleotide in the engineered host cell of the invention include, e.g., Herceptin®, polypeptide antigens from various pathogens such as disease causing bacteria or viruses (e.g., E. coli, P. aeruginosa, S. aureus, malaria, HIV, rabies virus, HBV, and cytomegalovirus), and other proteins such as lactoferrin, thioredoxin and beta-caseinvaccines.

Additional examples of therapeutic agents or proteins of interest include, but are not necessarily limited to tissue plasminogen activator (tPA), urokinase, streptokinase, neutropoesis stimulating protein (also known as filgastim or granulocyte colony stimulating factor (G-CSF)), thrombopoietin (TPO), growth hormone, emoglobin, insulinotropin, imiglucerase, sarbramostim, endothelian, soluble CD4, and antibodies and/or antigen-binding fragments (e.g., FAbs) thereof (e.g., orthoclone OKT-e (anti-CD3), GPIIb/IIa monoclonal antibody), ciliary neurite transforming factor (CNTF), granulocyte macrophage colony stimulating factor (GM-CSF), brain-derived neurite factor (BDNF), parathyroid hormone(PTH)-like hormone, insulinotrophic hormone, insulin-like growth factor-1 (IGF-1), platelet-derived growth factor (PDGF), epidermal growth factor (EGF), acidic fibroblast growth factor, basic fibroblast growth factor, transforming growth factor β, neurite growth factor (NGF), interferons (IFN) (e.g., IFN-α2b, IFN-α2a, IFN-αN1, IFN-β1b, IFN-γ), interleukins (e.g, IL-1, IL-2, IL-8), tumor necrosis factor (TNF) (e.g, TNF-α, TNF-β), transforming growth factor-α and -β, catalase, calcitonin, arginase, phenylalanine ammonia lyase, L-asparaginase, pepsin, uricase, trypsin, chymotrypsin, elastase, carboxypeptidase, lactase, sucrase, intrinsic factor, vasoactive intestinal peptide (VIP), calcitonin, Ob gene product, cholecystokinin (CCK), serotonin, and glucagon.

In some embodiments, a HH ribozyme variant of the invention is used to control expression of a target gene for regulating cell growth, differentiation or viability in cells transplanted into a subject. The expression constructs used in these methods expresses a target gene operably linked to a HH ribozyme variant of the invention. The target gene encodes a polypeptide that regulates the growth or other cellular processes of the cell. The level of expression of the polypeptide is dependent on the addition of an inducer (e.g., a morpholino oligomer). In these embodiments, binding of the inducer to the ribozyme suppresses activity of the cis-acting ribozyme, resulting in activation of expression of the polypeptide. Thus, expression of the polypeptide alters regulation of cell growth, differentiation or viability in the cells in a manner dependent on the administered inducer. As exemplification, these methods can be used to prevent the growth of hyperplastic or tumor cells, or even the unwanted proliferation of normal cells. The methods can also be used to induce the death of fat cells, to regulate growth and differentiation of stem cells, or to regulate activation of an immune response, or to regulate proliferation, apoptosis, or effector function of genetically engineered T cells (e.g. CAR-T cells) for cancer immune therapies.

In some embodiments, the target gene can encode an antigen of interest for the production of antibodies. Upon introducing the ribozyme-encoding expression construct into a subject, production of antibodies to the antigen encoded by the target gene can be controlled by the absence or presence of the inducer that specifically binds to the ribozyme fused to the antigen-expressing mRNA. In some other embodiments, the target gene can encode a reporter gene, e.g., fluorescent or luminescent reporter proteins such as GFP or luciferase, enzymatic reporters such as alkaline phosphatase, or colorimetric reporters such as lacZ. Expression of the reporter molecule can be used to correlate with and determine the amount of an antisense inducer that binds to the ribozyme.

In some preferred embodiments, the ribozyme-regulated expression constructs of the invention are employed in gene transfer or therapy. Retroviral vectors or recombinant retroviruses are widely employed in gene transfer in various therapeutic or industrial applications. For example, gene therapy procedures have been used to correct acquired and inherited genetic defects, and to treat cancer or viral infection in a number of contexts. The ability to express artificial genes in humans facilitates the prevention and/or cure of many important human diseases, including many diseases which are not amenable to treatment by other therapies. For a review of gene therapy procedures, see Anderson, Science 256:808-813, 1992; Nabel & Felgner, TIBTECH 11:211-217, 1993; Mitani & Caskey, TIBTECH 11:162-166, 1993; Mulligan, Science 926-932, 1993; Dillon, TIBTECH 11:167-175, 1993; Miller, Nature 357:455-460, 1992; Van Brunt, Biotechnology 6:1149-1154, 1998; Vigne, Restorative Neurology and Neuroscience 8:35-36, 1995; Kremer & Perricaudet, British Medical Bulletin 51:31-44, 1995; Haddada et al., in Current Topics in Microbiology and Immunology (Doerfler & Böhm eds., 1995); and Yu et al., Gene Therapy 1:13-26, 1994.

In some embodiments, the target gene to be expressed under the control of the ribozymes of the invention can be a gene that encodes a therapeutic polypeptide or agent noted above. For example, transfection of tumor suppressor gene p53 into human breast cancer cell lines has led to restored growth suppression in the cells (Casey et al., Oncogene 6:1791-7, 1991). In some other embodiments, the target gene operably linked to a HH ribozyme variant of the invention to can encode an enzyme. For example, the gene can encode a cyclin-dependent kinase (CDK). It was shown that restoration of the function of a wild-type cyclin-dependent kinase, p16INK4, by transfection with a p16INK4-expressing vector reduced colony formation by some human cancer cell lines (Okamoto, Proc. Natl. Acad. Sci. U.S.A. 91:11045-9, 1994). Additional embodiments of the invention encompass ribozyme controlled expression in target cells of cell adhesion molecules, other tumor suppressors such as p21 and BRCA2, inducers of apoptosis such as Bax and Bak, other enzymes such as cytosine deaminases and thymidine kinases, hormones such as growth hormone and insulin, and interleukins and cytokines. As exemplified herein, preferred target cells for the present invention are mammalian cells, e.g., cells of both human and non-human animals including vertebrates and mammals. In some embodiments, the target cells are cancer or tumor cells. Various cancer types are suitable for treatment with methods of the invention by introducing into the cancer cells an expression construct that can be turned on or off via the addition of an antisense oligonucleotide inhibitor of the ribozyme. In some other embodiments, the target cells are stem cells. The expression construct introduced into the cells can express a polypeptide that regulates differentiation and proliferation of stem cells. Stem cells suitable for practicing the invention include and are not limited to hematopoietic stem cells (HSC), embryonic stem cells or mesenchymal stem cells.

The invention provides engineered mammalian cells which express a target gene that is operably fused to a HH ribozyme variant sequence described herein. Using the ribozyme molecules or expression vectors of the invention, various mammalian cells can be employed for introducing an expression vector of the invention or by stably integrating the rDNA described herein into the host genome. Polynucleotides encoding the ribozymes or expression vectors can be introduced into an appropriate host cell (e.g., a mammalian cell such as 293T cell, N2a cell or CHO cell, or PBMC, or primary immune cells) by any means known in the art. The cells can transiently or stably express the introduced ribozyme containing target gene. Preferably, mammalian cells are used in these embodiments of the invention. Mammalian expression systems allow for proper post-translational modifications of expressed mammalian proteins to occur, e.g., proper processing of the primary transcript, glycosylation, phosphorylation and advantageously secretion of expressed product. Suitable cells include cells rodent, cow, goat, rabbit, sheep, non-human primate, human, and the like). Specific examples of cell lines include CHO, BHK, HEK293, N2a, VERO, HeLa, COS, MDCK, and W138. As exemplified herein, any convenient protocol may be employed for in vitro or in vivo introduction of the expression vector into the host cell, depending on the location of the host cell. In some embodiments, where the host cell is an isolated cell, the expression vector may be introduced directly into the cell under cell culture conditions permissive of viability of the host cell, e.g., by using standard transformation techniques.

Alternatively, where the host cell or cells are part of a multicellular organism, the targeting vector may be administered to the organism or host in a manner such that the expression vector is able to enter the host cell(s), e.g., via an in vivo or ex vivo protocol. By “in vivo,” it is meant in the target construct is administered to a living body of an animal. By “ex vivo” it is meant that cells or organs are modified outside of the body. Such cells or organs are typically returned to a living body. Techniques well known in the art for the transfection of cells can be used for the ex vivo administration of nucleic acid constructs. The exact formulation, route of administration and dosage can be chosen empirically. See e.g. Fingl et al., 1975, in The Pharmacological Basis of Therapeutics, Ch. 1 p 1). For example, nucleic acid constructs can be delivered with cationic lipids (Goddard, et al, Gene Therapy, 4:1231-1236, 1997; Gorman et al., Gene Therapy 4:983-992, 1997; Chadwick et al., Gene Therapy 4:937-942, 1997; Gokhale et al., Gene Therapy 4:1289-1299, 1997; Gao and Huang, Gene Therapy 2:710-722, 1995), using viral vectors (Monahan et al., Gene Therapy 4:40-49, 1997; Onodera et al., Blood 91:30-36, 1998), by uptake of “naked DNA”, and the like. In some other embodiments, the expression constructs of the invention can be introduced into the target cells via a liposome. Preferably, the liposome is composed of steroid, particularly a combined substance with phospholipid combined with cholesterol, particularly a combined substance with phospholipid with high phase transition temperature. The physical properties of liposomes depend on pH, ion strength and the existence of divalent cations.

Pharmaceutical preparations or compositions are typically employed in the practice of the various therapeutic embodiments of the invention. The pharmaceutical preparations contain a HH ribozyme variant of the invention or its coding sequence, or an expression construct harboring the ribozyme-coding sequence. In some embodiments, a target gene sequence is operably linked to the ribozyme-coding sequence in the expression construct as described herein. In addition, the pharmaceutical compositions of the invention can also contain a pharmaceutically acceptable carrier suitable for administration to a human or non-human subject. The pharmaceutically acceptable carrier can be selected from pharmaceutically acceptable salts, ester, and salts of such esters.

### EXAMPLES

The following examples are provided to further illustrate the invention but not to limit its scope. Other variants of the invention will be readily apparent to one of ordinary skill in the art and are encompassed by the appended claims.

### Example 1. Hammerhead Ribozyme Variants with Improved Activities

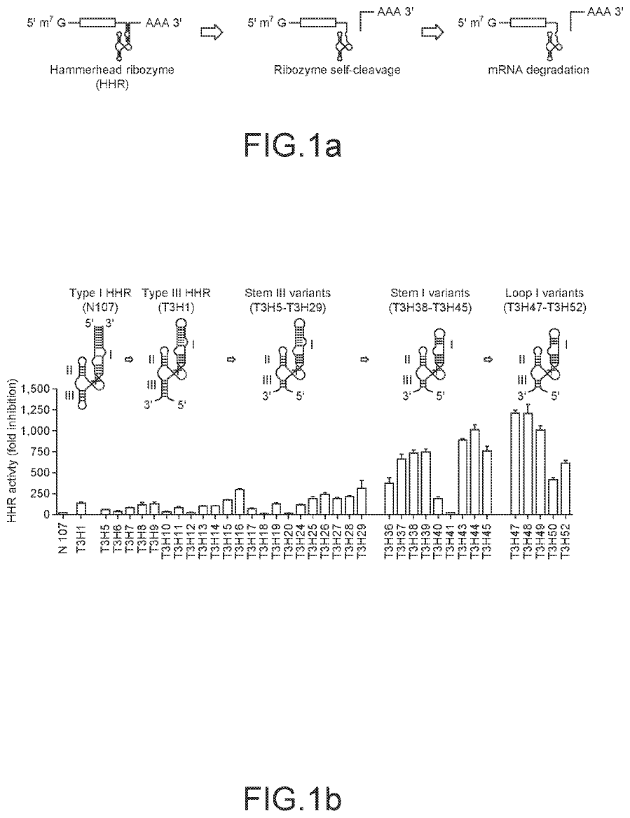

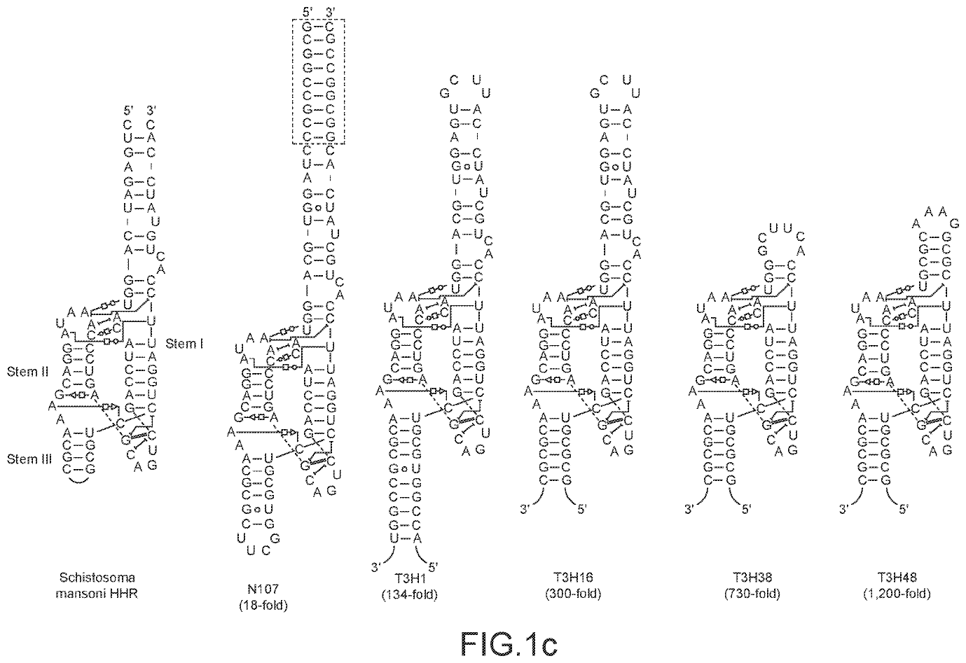

To engineer RNA effector domains with wider dynamic ranges, we selected a modified Schistosoma mansoni HH ribozyme, N107, as a starting point (Yen et al., Nature 431:471-476, 2004). We rationally designed a panel of ribozyme variants, introduced them into the 3′ UTR of a Gaussia luciferase (Gluc) gene, and tested their catalytic activity in a cell-culture reporter inhibition assay (FIG. 1a). A functional ribozyme's catalytic activity was determined as its fold inhibition in Gluc expression relative to the expression observed with a corresponding catalytically inactive mutant.

The inactive mutant contains only one nucleotide substitution in the highly conserved enzymatic core of the hammerhead ribozyme. Using sequence of T3H16 (SEQ ID NO:9) below as an example, residues forming the enzymatic core in its tertiary structure are underlined. The inactive ribozyme variant was generated by replacing an adenine residue (italicized) in the core towards the end of the sequence with a guanine residue. Inactive variants of all other tested ribozymes were also made by introducing the A→G mutation to the same site of the enzymatic core.

T3H16:
(SEQ ID NO: 9)
gcgcgtcctggattccactgctatccattcgtgaggtgcaggtacatcc
agctgacgagtcccaaataggacga [symbol] acgcgc

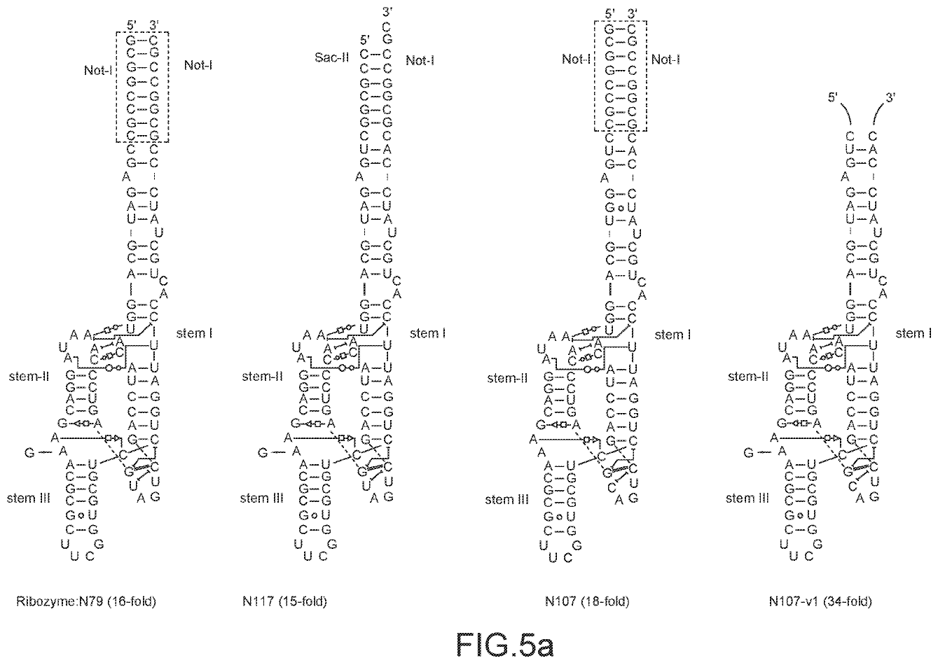

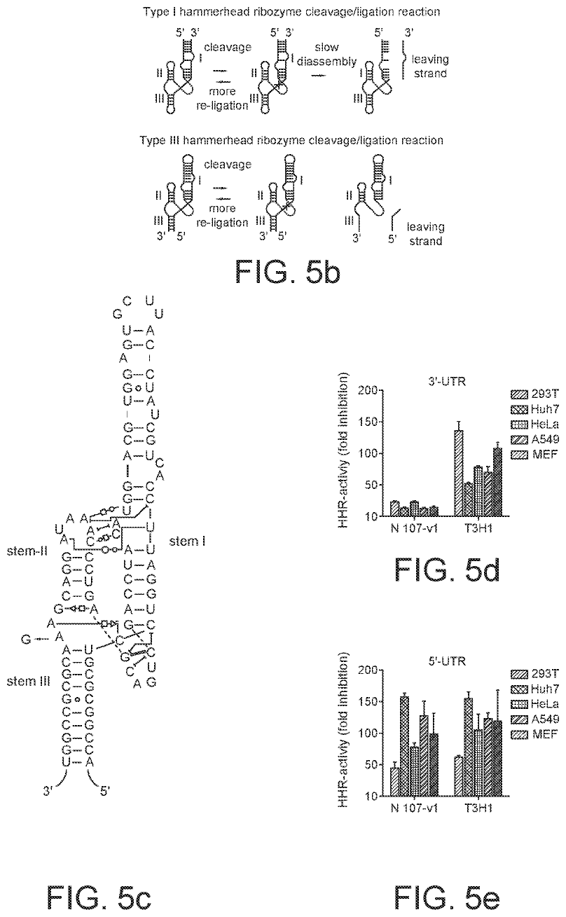

The results indicate that the N107 ribozyme, a type I HH ribozyme, affords only an 18-fold inhibition of GLuc expression in 293T cells (FIG. 1b, and FIG. 5a). We reasoned that base-pairing interactions holding the long leaving strand of stem I and the tertiary interactions between this strand and loop II may result in a relatively slow disassembly of the cleaved type I ribozyme, allowing translation to continue and facilitating re-ligation of the cleaved substrate strand (FIG. 5a-b). In contrast, a type III ribozyme can have a shorter leaving strand and fewer tertiary interactions between its leaving strand and the remainder of the ribozyme. Thus, type III ribozymes may disassemble more quickly after cleavage, preventing translation or re-ligation. Indeed, the ribozyme fold-inhibition activity significantly increased when N107 was converted to a type III ribozyme, T3H1, and placed at the 3′ UTR (FIG. 1b and FIG. 5c-e).

We then hypothesized that adjusting the stem-III annealing energy could accelerate disassembly without sacrificing cleavage efficiency (FIG. 6a). Accordingly, we tested ribozyme variants with stem-III regions of varying lengths or with different potential inter-strand base stackings. Two of these stem-III variants, T3H16 and T3H29, inhibited Gluc expression in 293T cells by ˜300-fold and significantly outperformed all other stem-III variants tested (FIG. 1b and FIG. 6b-h). When ribozyme activities of these stem-III variants were plotted against their calculated stem-III annealing energies, a peak of approximately −9 kcal/mol was observed, corresponding to the annealing energies of both T3H16 and T3H29 (FIG. 6i). Tertiary interactions between loop II and a bulge on stem I enhance HH ribozyme activity by three orders of magnitude (FIG. 7a). We therefore modified stem I of T3H16 to facilitate these tertiary interactions and improve ribozyme activity. We observed, as expected, that changes designed to destabilize the bulge I structure (T3H40 and T3H41) or directly impair these tertiary interactions (T3H54, T3H56, and T3H57) impaired ribozyme activity dramatically (FIG. 7b-d). In contrast, changes that could stabilize the bulge I significantly improved the ribozyme activity to 1000-fold (T3H44; FIG. 1b and FIG. 7b). Finally, by changing the T3H44 loop I nucleotides to more stable tetraloops (see, e.g., Ennifar et al., J Mol Biol 304:35-42, 2000; and Fiore et al., Q Rev Biophys 46:223-264, 2013), ribozyme activity increased to ˜1200-fold (FIG. 1b and FIG. 7e).

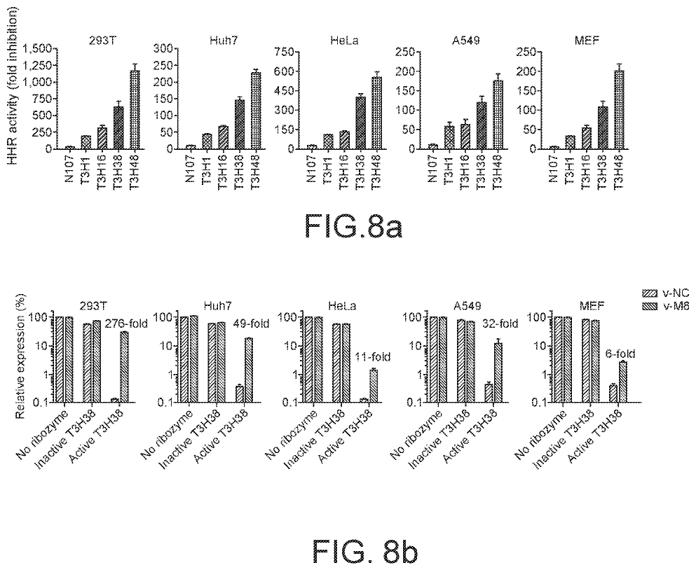

Thus, by converting HH ribozyme N107 to a type III ribozyme, and by optimizing its stem III, stem I, and stem-I loop, ribozyme activity increased from its original 18-fold to ˜1200-fold. The most efficient of these ribozymes, T3H48, was 60- to 80-fold more active than the type I ribozymes N107 and N117, and 92-fold more active than sTRSV ribozyme, a well-characterized type III ribozyme (FIG. 7f-j). This increase in activity was observed across multiple cell lines, indicating these optimizations were not cell-type dependent (FIG. 8a). Sequences and secondary structures of the native Schistosoma mansoni ribozyme, the engineered Schistosoma mansoni HH ribozyme N107, and several key milestone variants in this optimization process are shown in FIG. 1c.

### Example 2. Modulating Gene Expression by Inhibiting Ribozyme Activity with Morpholino, an RNase H-Independent Antisense Oligonucleotide

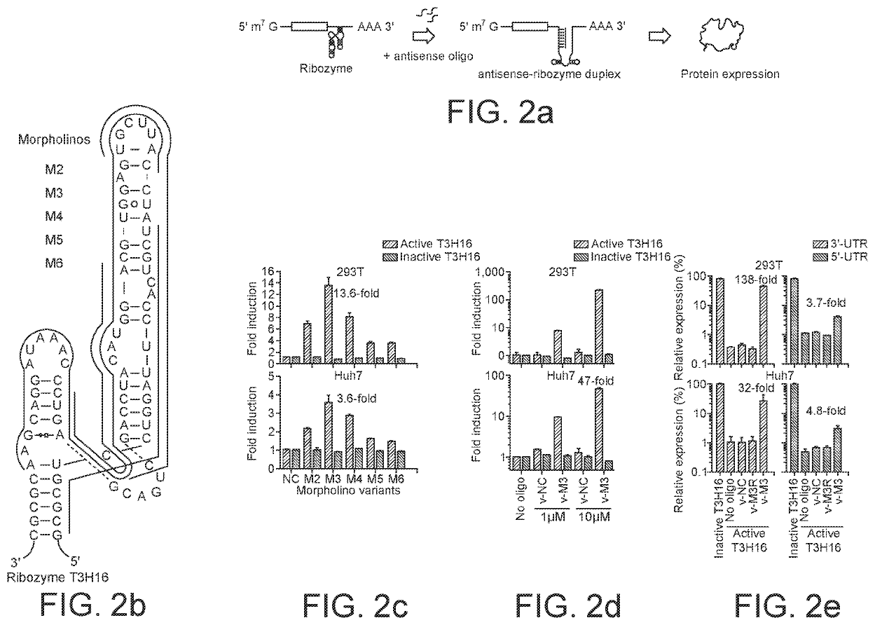

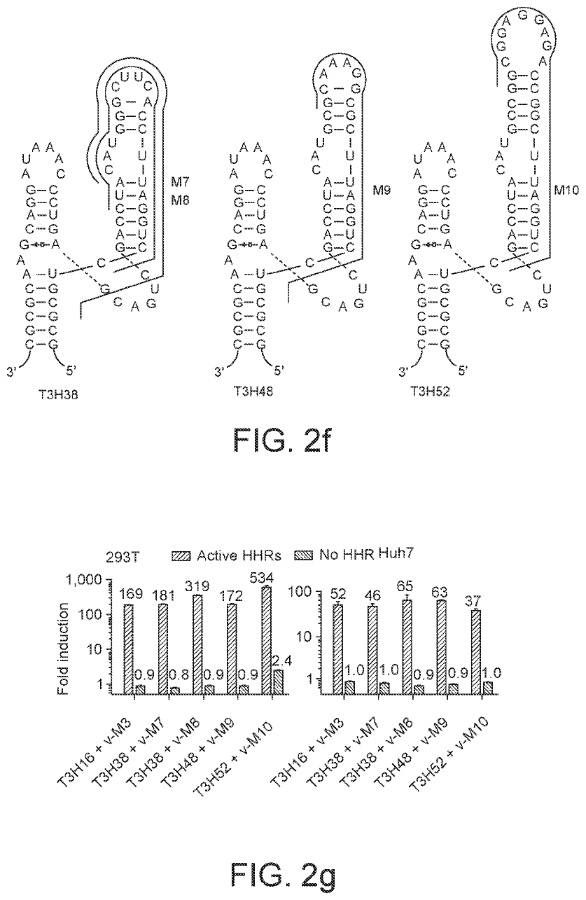

Ribozyme activity can be regulated by complementary phosphorodiamidate morpholino oligomers (FIG. 2a). These morpholinos are a class of RNase H-independent antisense oligonucleotides that have been widely used to control splicing in scientific and clinical contexts, and have been approved for use in humans. We first tested a panel of morpholinos complementary to different regions of T3H16 ribozyme for their ability to interfere with the activity of T3H16 ribozyme and thus induce gene expression. The M3 morpholino, which targets 5′ portion of the stem I, most efficiently interfered with T3H16 placed at the 3′ UTR of Gluc gene, inducing ˜14-fold expression of Gluc in 293T cells, whereas the same morpholino had no effect on an inactive ribozyme control (FIGS. 2b and c). We then tested an octa-guanidine dendrimer-coupled form (Morcos et al., Biotechniques 45:613-624, 2008) of M3 oligo, v-M3, with the ‘v’ denoting this dendrimer modification. v-M3 induced Gluc expression up to 200-fold, an effect that was dose dependent and markedly greater when T3H16 was placed at the 3′ UTR than at the 5′ UTR (FIGS. 2d and e), consistent with the ability of morpholinos to inhibit translation when targeting the 5′ UTR. We speculate that M3 best regulates T3H16 because it targets the 5′ region of the ribozyme, a region accessible during transcription before the ribozyme assembles. By contrast, morpholinos that target the 3′ region of the ribozyme must compete with the already synthesized 5′ strand. We accordingly assayed the ability of 5′-targeting morpholinos similar to M3 to regulate more efficient ribozyme variants (T3H38, T3H48) and a variant with a larger and more accessible stem-I loop (T3H52) (FIG. 2f). The most efficient regulation was afforded by v-M8, which specifically induced gene expression controlled by ribozyme T3H38 by ˜300-fold in 293T cells (FIGS. 2g and 8b). Sequences of the various morpholino oligonucleotides used in these studies are shown below.

Sequences of the oligonucleotide in vivo-morpholinos M2-M10:

M2:
(SEQ ID NO: 38)
gatagcagtggaatccaggacgcac
M3:
(SEQ ID NO: 39)
cgaatggatagcagtggaatccagg
M4:
(SEQ ID NO: 40)
ctggatgtacctgcacctcacgaat
M5:
(SEQ ID NO: 41)
ggactcgtcagctggatgtacctgc
M6:
(SEQ ID NO: 42)
ttcgtcctatttgggactcgtcagc
M7:
(SEQ ID NO: 43)
gatgtacccgaagtggaatccagg
M8:
(SEQ ID NO: 44)
gtacccgaagtggaatccaggacgc
M9:
(SEQ ID NO: 45)
cgtttccgcgaatccaggacgc
M10:
(SEQ ID NO: 46)
gccgcctcctctggccgaatccagg

### Example 3. Controlling Gene Expression In Vivo

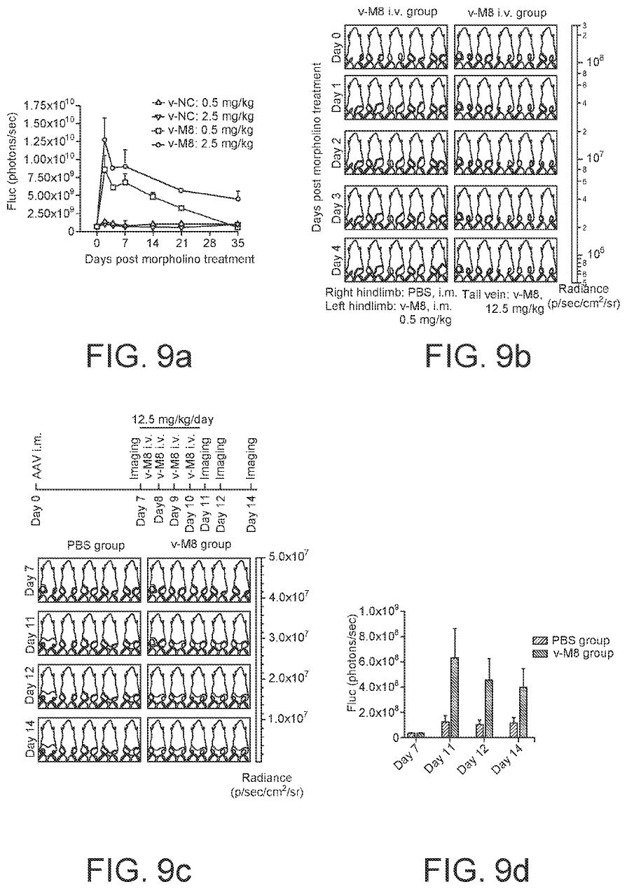

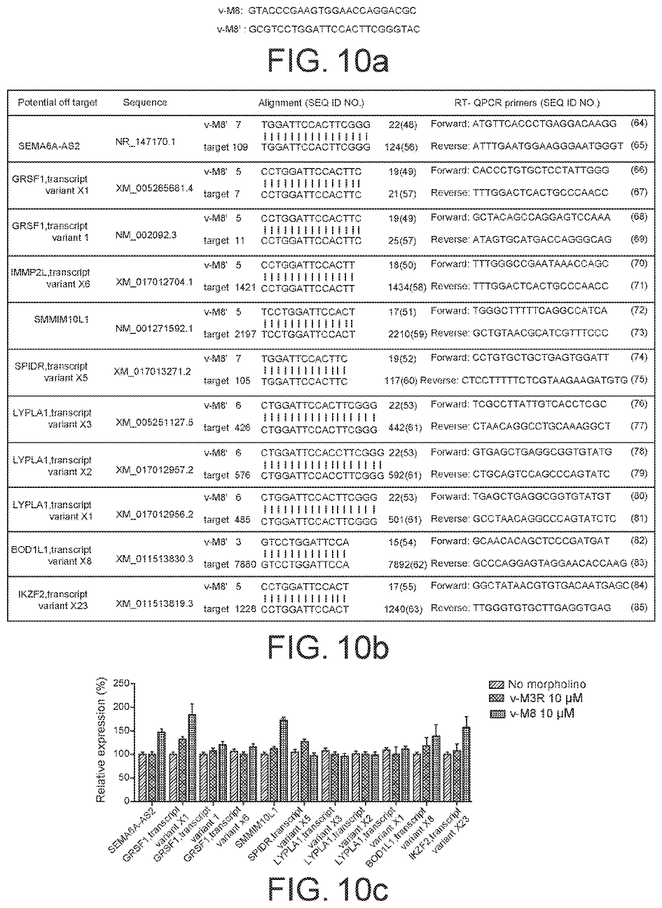

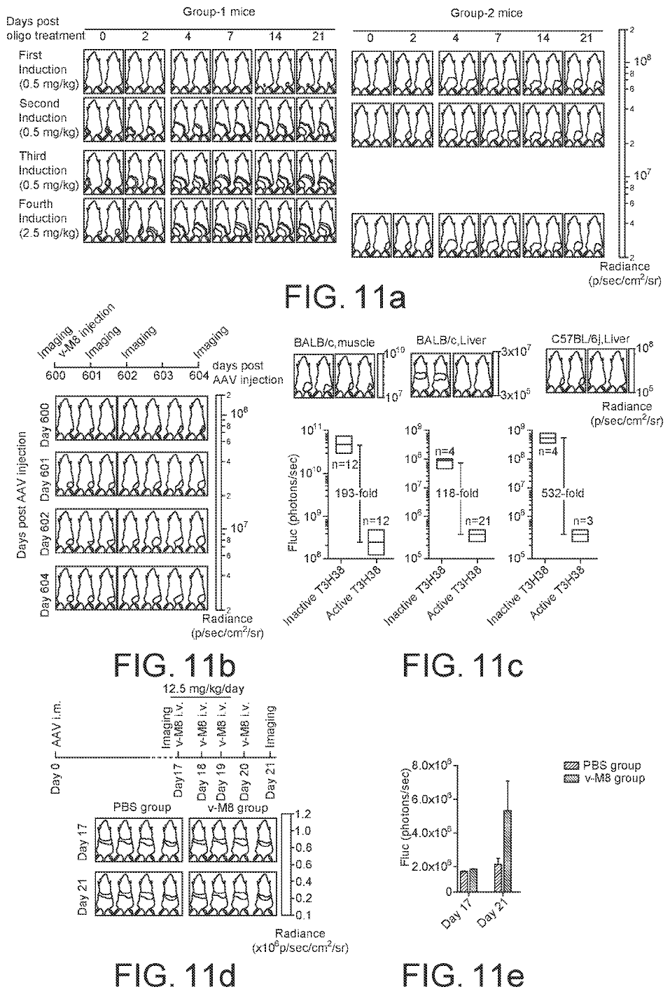

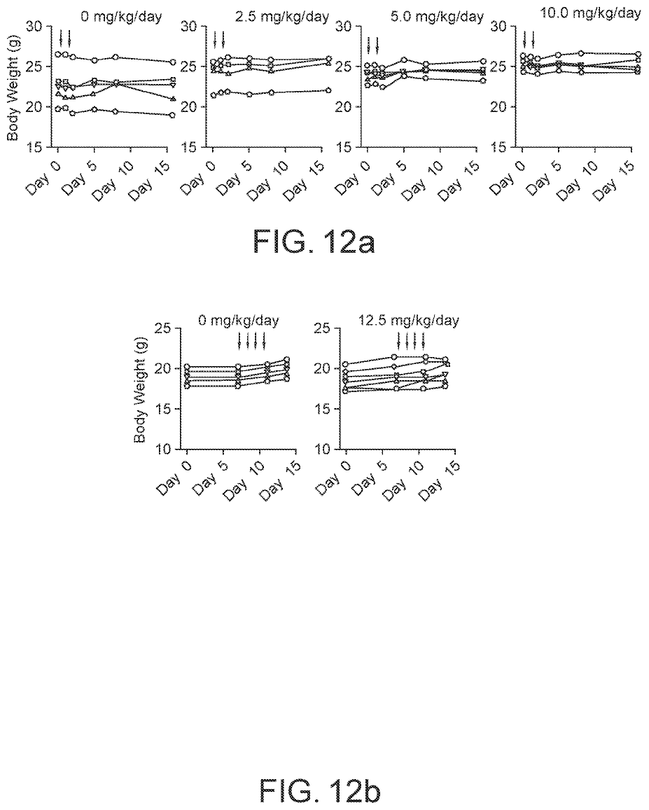

The exceptionally broad regulatory range of T3H38 by v-M8 suggested that this combination could provide useful in vivo control of an AAV transgene. We opted to assess this system in skeletal muscle because its slow turnover and extensive vascularization makes it a useful target tissue for gene therapies that express secretory therapeutic proteins to treat a range of human diseases. Mouse gastrocnemius muscles were accordingly injected with an AAV1 vector expressing a firefly luciferase (Fluc) gene with the T3H38 ribozyme at its 3′ UTR (FIG. 3a). We observed efficient and dose-dependent induction of luciferase expression by the functional morpholino, v-M8, but not the control morpholino, v-NC (FIGS. 3b, 3c, and 9a). Importantly, intramuscular injection of morpholino only induced luciferase expression from the morpholino-injected hindlimb but not the opposite hindlimb. In addition, this locally induced expression was markedly more efficient than the expression induced by systemic injections of 25-fold higher morpholino dose (FIGS. 3d and 9b-d). Of note, v-M8 showed modest off-target effect on several ubiquitously expressed genes (FIG. 10). These data together indicate that local administration of morpholino for transgene regulation is more specific, more efficient, and safer for potential medical applications. Moreover, with local induction, luciferase expression could be repeatedly induced by more than 100-fold over a period of at least 43 weeks (FIGS. 3e and FIG. 11a). Reporter expression in these mice remained inducible even 20 months after AAV injection (FIG. 11b). Liver is another attractive target organ for gene therapy. Although the T3H38 ribozyme is similarly efficient in mouse liver and muscle tissue, we observed that our switch could regulate AAV-delivered transgene expression in liver by only 3-fold (FIGS. 11c-e). The higher regulatory range in muscle is likely a result of a higher effective concentration in muscle tissue when the morpholino is locally administered. More efficient regulation of liver expression may be achieved by replacing the octa-guanidine dendrimer in v-M8 with liver-targeting N-acetylgalactosamine (see, e.g., Huang Y. Mol Ther Nucleic Acids. 2017. 6:116-132). Collectively, our data suggest a morpholino-regulated ribozyme-based on-switch is a feasible means of regulating expression of biologics in vivo.

To highlight one medically useful application of this system, we sought to control the expression of AAV-delivered erythropoietin (Epo), a commonly administered biologic for the treatment of anemia associated with chronic kidney disease. Mouse gastrocnemius muscles were injected with an AAV1 vector delivering the murine Epo gene with the T3H38 ribozyme at its 3′ UTR (FIG. 4a). The v-M8 morpholino was administered twelve days later to the same site. v-M8 induced Epo expression and hematocrit levels to those observed with an Epo transgene incorporating a catalytically inactive T3H38 (FIG. 4b). Importantly, Epo concentrations remained elevated over at least two weeks with an induction half-life of 122 hours. In contrast, the half-life of recombinant Epo protein was ˜3 hours, consistent with previous studies (Lee et al., Biochem. Biophys. Res. Commun. 339:380-385, 2006). Morpholino-induced Epo peaked at 15,000 pg/ml, 100-fold higher than physiological concentrations, and far higher than what would be optimal for Epo treatment. We thus injected mice intramuscularly with lower doses of AAV (5×10⁹ and 2×10⁹ genome copies) and also varied the dose of morpholino (0, 0.1, 0.5 and 2.5 mg/kg). We observed morpholino- as well as AAV-dose-dependent induction of erythropoietin expression and hematocrit levels (FIGS. 4c-f). When mice were injected with 5×10⁹ genome copies of AAV, the lowest tested dose of morpholino (0.1 mg/kg) induced Epo expression at close-to-physiological concentrations for over a week (FIG. 4c). Interestingly, when the AAV dose was further lowered to 2×10⁹ genome copies, morpholino-induced Epo expression peaked at only 110 pg/ml, a concentration lower than physiological levels, but induced hematocrit remained elevated more than 4 weeks (FIGS. 4e and f). FIGS. 4g and h summarize Epo induction values induced by different doses of morpholino and AAV, respectively. Thus, sustained expression of physiologically relevant levels of Epo can be induced from a T3H38 ribozyme-regulated AAV transgene.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

All publications, databases, GenBank sequences, patents, and patent applications cited in this specification are herein incorporated by reference as if each was specifically and individually indicated to be incorporated by reference.

What is claimed is:

1. A type III variant of a type I cis-acting hammerhead ribozyme, comprising the 5′ and 3′ ends of its sequence in stem III.

2. The hammerhead ribozyme variant of claim 1, wherein the type I cis-acting hammerhead ribozyme is ribozyme N107, N117, or a natural Schistosoma mansoni hammerhead ribozyme.

3. The hammerhead ribozyme variant of claim 1, comprising a sequence (5′-3′) accgg ugcgu ccugga uucca cugcu aucca uucgu gaggu gcagg uacau ccagc ugacg agucc caaau aggac gaaac gcgcc ggu (SEQ ID NO:8).

4. The hammerhead ribozyme variant of claim 1, further comprising one or more additional modifications of the sequence of the type I cis-acting hammerhead ribozyme, wherein the additional modification is (a) optimization of stem III of the resulting type III ribozyme or (b) modifications of stem I and loop Ito facilitate formation of the “UAC” bulge at stem I and stabilize the tertiary interactions between the “UAC” bulge and loop II.

5. The hammerhead ribozyme variant of claim 4, wherein the optimized stem III comprises from about 4 to about 8 pair pairs, and has a calculated annealing energy of about −9 kcal/mol.

6. The hammerhead ribozyme variant of claim 5, wherein the two sequences on the opposite sides of the optimized stem III respectively contain residues (5′-3′/3′-5′) acgcgc/ugcgcg or acgccg/ugcggc.

7. The hammerhead ribozyme variant of claim 5, comprising a sequence (5′-3′) as shown in SEQ ID NO:9 or SEQ ID NO:10.

8. The hammerhead ribozyme variant of claim 4, wherein the two sequences on the opposite sides of the modified stem I, immediately 5′ to the “UAC” bulge, are perfectly matched.

9. The hammerhead ribozyme variant of claim 8, wherein the two sequences on the opposite sides of the modified stem I, immediately 5′ to the “UAC” bulge, respectively contain residues (5′-3′/3′-5′) cc/gg, cgc/gcg, cgcg/gcgc, or cgcgc/gcgcg.

10. The hammerhead ribozyme variant of claim 4, wherein the altered loop I comprises a sequence (5′-3′) auuc, uucg, gaaa, ucac, or agaggaggc.

11. The hammerhead ribozyme variant of claim 4, comprising both an optimized stem III and a modified stem I.

12. The hammerhead ribozyme variant of claim 11, comprising a sequence (5′-3′) shown in any one of SEQ ID NOs:11-17.

13. The hammerhead ribozyme variant of claim 4, comprising an optimized stem III, a modified stem I and an altered loop I.

14. The hammerhead ribozyme variant of claim 13, comprising a sequence (5′-3′) shown in any one of SEQ ID NOs:18-22.

15. An expression vector, comprising a target gene sequence that is operably fused to an hammerhead ribozyme-coding sequence, wherein the hammerhead ribozyme-coding sequence is inserted into the target gene at its 3′-UTR or 5′-UTR, and wherein the hammerhead ribozyme is a type III variant of a type I cis-acting hammerhead ribozyme that comprises the 5′ and 3′ ends of its sequence in Stem III.

16. The expression vector of claim 15, further comprising one or more transcriptional regulatory sequences that regulate transcription of the target gene in a mammalian cell.

17. The expression vector of claim 15, which is a DNA vector.

18. The expression vector of claim 15, wherein the hammerhead ribozyme comprises one or more additional modifications of the sequence of the type I cis-acting hammerhead ribozyme, wherein the additional modification is (a) optimization of stem III to have a calculated annealing energy of about −9 kcal/mol or (b) modifications of stem I and loop I to facilitate formation of the “UAC” bulge at stem I and stabilize the tertiary interactions between the “UAC” bulge and loop II.

19. The expression vector of claim 18, wherein the hammerhead ribozyme comprises a sequence (5′-3′) shown in any one of SEQ ID NOs:9-22.

20. The expression vector of claim 15, wherein the target gene encodes erythropoietin (Epo).

21. An engineered mammalian cell harboring the expression vector of claim 15.

22. A method for inducing expression of a target gene in a cell, comprising (a) constructing an expression vector of claim 15, (b) introducing the expression vector into the cell, and (c) contacting the cell with an RNase H-independent antisense oligonucleotide that is complementary to the hammerhead ribozyme-coding sequence in the expression vector;
thereby inducing expression of the target gene in the cell.

23. The method of claim 22, wherein the RNase H-independent antisense oligonucleotide is a morpholino oligonucleotide.

24. The method of claim 22, wherein the cell is a mammalian cell.

25. The method of claim 22, wherein the expression vector is an adeno-associated virus (AAV) vector.

26. The method of claim 22, wherein the hammerhead ribozyme encoded by the vector comprises one or more additional modifications of the sequence of the type I cis-acting hammerhead ribozyme, wherein the additional modification is (a) optimization of stem III to have a calculated annealing energy of about −9 kcal/mol or (b) modifications of stem I and loop I to facilitate formation of the “UAC” bulge at stem I and stabilize the tertiary interactions between the “UAC” bulge and loop II.

27. The method of claim 26, wherein the hammerhead ribozyme encoded by the vector comprises a sequence (5′-3′) shown in any one of SEQ ID NOs:9-22.

28. The method of claim 22, wherein the target gene encodes erythropoietin (Epo).

29. The method of claim 22, wherein the RNase H-independent antisense oligonucleotide is a modified morpholino.

30. The method of claim 29, wherein the modified morpholino is an octa-guanidine dendrimer-coupled morpholino.

31. The method of claim 22, wherein the RNase H-independent antisense oligonucleotide comprises an oligonucleotide sequence as shown in any one of SEQ ID NOs:38-46.

32. The method of claim 22, wherein the cell is present in a subject in need of the polypeptide encoded by the target gene.

33. The method of claim 22, wherein the cell is obtained from the subject prior to introduction of the expression vector into the cell.

34. The method of claim 33, further comprising, subsequent to introducing the expression vector into the cell, reintroducing the cell into the subject.

35. The method of claim 33, wherein the RNase H-independent antisense oligonucleotide is administered to the subject subsequent to reintroducing the cell into the subject.
